SEQUENCE LISTING (3 sequences)

SEQ ID NO: 1 - protein, 7 aa, Bovine serum albumin tryptic peptide
  VARIANT (1)..(2): Y = N, NH2, NO2
YLYEIAR

SEQ ID NO: 2 - protein, 7 aa, Artificial Sequence
  VARIANT (5)..(6): Y = NO2, N, NH2
AAFGYAR

SEQ ID NO: 3 - protein, 13 aa, Bovine serum albumin tryptic peptide
LGEYGEQNALIVR

(19) United States
(12) Patent Application Publication
(10) Pub. No.: US 2003/0165983 A1
(43) Pub. Date: Sep. 4, 2003

(54) Proteomic determination of protein nitrotyrosine modifications using mass spectrometry

(76) Inventors: Bradford W. Gibson, Berkeley, CA (US); Soumitra S. Ghosh, San Diego, CA (US); Robert E. Davis, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/847,522

(22) Filed: May 1, 2001

Related U.S. Application Data
(60) Provisional application No. 60/201,177, filed on May 2, 2000.

(51) Int. Cl. G01N 33/53; H01J 49/00

(52) U.S. Cl. 435/7.1; 250/282

(57) ABSTRACT

Compositions and methods are provided for identifying oxidative modifications of proteins by mass spectrometric analysis, including MALDI-TOF MS, of protein and peptide fractions of biological samples to determine specific occurrences of nitrotyrosine at amino acid sequence and proteomic levels. Diagnostic methods for diseases characterized by elevated free radicals and oxidative stress, and screening assays for therapeutic agents useful in treating such diseases, are also disclosed.

### CROSS REFERENCE TO RELATED APPLICATION

This application claims the benefit of U.S. Provisional Application No. 60/201,177, filed May 2, 2000, which is hereby incorporated by reference in its entirety.

### STATEMENT OF GOVERNMENT INTEREST

The Regents of the University of California may have certain rights in this invention under the Biotechnology Star Project Research Agreement No.S97-05.

### TECHNICAL FIELD

The present invention relates generally to compositions and methods for identifying oxidative modification of proteins and peptides. More specifically, the invention is directed to determining the presence of nitrotyrosine in proteins from biological samples and in particular, to proteomic profiling of proteins based on nitrotyrosine content.

### BACKGROUND OF THE INVENTION

Free radical production in biological systems is known to result in the generation of reactive species that can chemically modify molecular components of cells and tissues. Such modifications can alter or disrupt structural and/or functional properties of these molecules, leading to compromised cellular activity and tissue damage. Although mitochondria are a primary source of free radicals in biological systems (see, e.g., Murphy et al., 1998 in Mitochondria and Free Radicals in Neurodegenerative Diseases, Beal, Howell and Bodis-Wollner, Eds., Wiley-Liss, New York, pp. 159-186 and references cited therein), free radical production may also arise in extramitochondrial locales and can contribute to pathological processes regardless of the particular subcellular source site. For example, numerous intracellular biochemical pathways that lead to the formation of radicals through production of metabolites such as hydrogen peroxide, nitric oxide or superoxide radical via reactions catalyzed by enzymes such as flavin-linked oxidases, superoxide dismutase (SOD) or nitric oxide synthetase, are known in the art, as are methods for detecting such radicals (see, e.g., Kelver, 1993 Crit. Rev. Toxicol. 23:21; Halliwell B. and J. M. C. Gutteridge, Free Radicals in Biology and Medicine, 1989 Clarendon Press, Oxford, UK; Davies, K. J. A. and F. Ursini, The Oxygen Paradox, Cleup Univ. Press, Padova, IT). Altered mitochondrial function, such as failure at any step of the mitochondrial electron transport chain (ETC), may also lead to the generation of highly reactive free radicals. Thus, free radicals generated in biological systems, including free radicals resulting from altered mitochondrial function or from extramitochondrial sources, include reactive oxygen species (ROS), for example, superoxide, peroxynitrite and hydroxyl radicals, and potentially other reactive species that may be toxic to cells.

There are a variety of methods for detecting a free radical that are known in the art, where selection of such a method depends on the particular radical of interest. Typically, a level of free radical production in a biological sample may be determined according to methods including detection and/or measurement of: glycoxidation products including pentosidine, carboxymethylysine and pyrroline; lipoxidation products including glyoxal, malondialdehyde and 4-hydroxynonenal; thiobarbituric acid reactive substances (TBARS; see, e.g., Steinbrecher et al., 1984 Proc. Nat. Acad. Sci. USA 81:3883; Wolff, 1993 Br. Med. Bull. 49:642) and/or other chemical detection means such as salicylate trapping of hydroxyl radicals (e.g., Ghiselli et al., 1998 Meths. Mol. Biol. 108:89; Halliwell et al., 1997 Free Radic. Res. 27:239) or specific adduct formation (see, e.g., Mecocci et al. 1993 Ann. Neurol. 34:609; Giulivi et al., 1994 Meths. Enzymol. 233:363) including malondialdehyde formation, protein nitration or nitrosylation, DNA oxidation including mitochondrial DNA oxidation, 8′-OH-guanosine adducts (e.g., Beckman et al., 1999 Mutat. Res. 424:51), protein oxidation, protein carbonyl modification (e.g., Baynes et al., 1991 Diabetes 40:405; Baynes et al., 1999 Diabetes 48:1); electron spin resonance (ESR) probes; cyclic voltametry; fluorescent and/or chemiluminescent indicators (see also e.g., Greenwald, R. A. (ed.), Handbook of Methods for Oxygen Radical Research, 1985 CRC Press, Boca Raton, Fla.; Acworth and Bailey, (eds.), Handbook of Oxidative Metabolism, 1995 ESA, Inc., Chelmsford, Mass.; Yla-Herttuala et al., 1989 J. Clin. Invest. 84:1086; Velazques et al., 1991 Diabetic Medicine 8:752; Belch et al., 1995 Int. Angiol. 14:385; Sato et al., 1979 Biochem. Med. 21:104; Traverso et al., 1998 Diabetologia 41:265; Haugland, 1996 Handbook of Fluorescent Probes and Research Chemicals—Sixth Ed., Molecular Probes, Eugene, Oreg., pp. 483-502, and references cited therein).

For example, by way of illustration and not limitation, oxidation of the fluorescent probes dichlorodihydrofluorescein diacetate and its carboxylated derivative carboxydichlorodihydrofluorescein diacetate (see, e.g., Haugland, 1996, supra) may be quantified following accumulation in cells, a process that is dependent on, and proportional to, the presence of reactive oxygen species (see also, e.g., Molecular Probes On-line Handbook of Fluorescent Probes and Research Chemicals, at http://www.probes.com/handbook/toc.html). Other fluorescent detectable compounds that may be used for detection of free radical production include but are not limited to dihydrorhodamine and dihydrorosamine derivatives, cis-parinaric acid, resorufin derivatives, lucigenin and any other suitable compound that may be known to those familiar with the art.

Oxidative damage to proteins, such as protein modification that results from reactive free radical activity in biological systems, is an underlying feature in the pathogenesis of a number of diseases, including Alzheimer's disease (AD), diabetes mellitus, Parkinson's disease, amyotrophic lateral sclerosis (ALS), atherosclerosis and other degenerative and inflammatory diseases. For example, free radical mediated damage may inactivate one or more of the myriad proteins of the mitochondrial ETC and in doing so, may uncouple the mitochondrial chemiosmotic mechanism responsible for oxidative phosphorylation and ATP production. Free radical mediated damage to mitochondrial functional integrity is also just one example of multiple mechanisms associated with altered mitochondrial function that may result in collapse of the electrochemical potential maintained by the inner mitochondrial membrane. Methods for detecting changes in the inner mitochondrial membrane potential are described, for instance, in U.S. patent application Ser. No. 09/161,172.

In humans and other living systems, the nitration of tyrosine residues in proteins is the result of oxidative damage mediated by reactive nitrogen-containing species. Mounting evidence suggests that 3-nitrotyrosine is an important biomarker for many diseases where oxidative stress is considered a key component, such as Alzheimer's disease (AD), Parkinson's disease, amyotrophic lateral sclerosis (ALS) or even the aging process itself. An important mediator of oxidative stress is peroxynitrite, which is formed by the reaction of nitric oxide and superoxide. The reaction forming peroxynitrite occurs at an extremely fast rate of 6.7×10⁹ sec⁻¹, which is 3-fold faster than the rate of dismutation of superoxide by its scavenging enzyme, superoxide dismutase (SOD). Peroxynitrite is highly reactive and nitrates proteins, lipids and DNA. The most prevalent modification of proteins by peroxynitrite is the nitration of tyrosine residues to 3-nitrotyrosine, a process believed to result from a random process that is a secondary consequence of oxidative stress and the production of peroxynitrite radicals (Beckman 1996 Chem. Res. Toxicol. 9:836-844; Maruyama et al., 1996 J. Chromatogr. B. Biomed. Appl. 676:153-158; Scheme 1). The formation of 3-nitrotyrosine is often used as a biomarker of peroxynitrite generation in vivo.

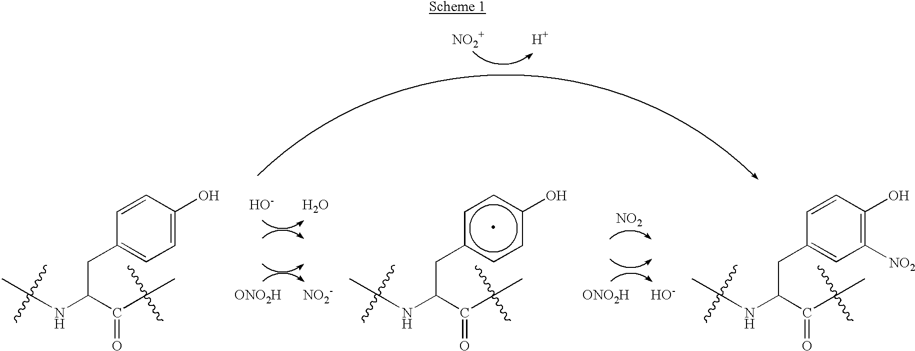

Depending on the particular protein affected by peroxynitrite-mediated modification, nitration of tyrosines can affect critical biochemical pathways by altering enzymatic activities and signal transduction processes. For example, tyrosine modification inhibits the reactivity of glutamine synthetase (Berlett et al., 1996 Proc. Nat. Acad. Sci. USA 93:1776) and disables the ability of tyrosine kinases to phosphorylate tyrosines, which is a critical event in many cell signaling pathways and in cell regulation (Kong et al., 1996 Proc. Nat. Acad. Sci. USA 93:3377). As another example, inactivation of human manganese-SOD by peroxynitrite is caused by exclusive nitration of Tyr-34 to 3-nitrotyrosine (Yamakura et al., 1998 J. Biol. Chem. 273:14085; MacMillan et al., 1998 Biochem. 37:1613). This residue is located near manganese and is a substrate O₂ gateway in Mn-SOD. According to non-limiting theory, inactivation of Mn-SOD by nitration at Tyr-34 decreases SOD radical scavenging activity, permitting generation of increased levels of peroxynitrite and thereby leading to increased protein tyrosine nitration. Similarly, inducible nitric oxide synthase levels in several disease states may lead to increased levels of nitric oxide, which may directly or indirectly (e.g., through peroxynitrite production) contribute to tyrosine nitration. Thus, protein tyrosine nitration may result in serious consequences for a cell if specific proteins or enzymes are modified that can further lead to cell damage, possibly resulting in programmed cell death, or apoptosis, under the most extreme situations. Immununohistological studies using various anti-nitrotyrosine antibodies have found elevated levels generally of proteins containing nitrotyrosine in several disease states, including AD, ALS and acute lung diseases, but have failed specifically to identify particular oxidatively modified protein species.

Accordingly, 3-nitrotyrosine has been identified as an anomalous amino acid derivative that signifies the presence of conditions permitting oxidative protein damage, and the formation of 3-nitrotyrosine can significantly alter the structure and/or impair the activity of a protein containing tyrosine residues in functionally significant positions. However, even where oxidative protein damage has been linked to a number of degenerative diseases, no specific proteins or collections of proteins that have been modified to contain 3-nitrotyrosine, nor specific tyrosine residues within such proteins that are preferentially susceptible to oxidative nitration, have yet been linked to particular disease processes.

For instance, highly variable levels of nitrotyrosine have been measured directly from oxidatively nitrated free tyrosine, and also from in vitro hydrolyzed protein, using both high performance liquid chromatography with electrochemical detection (HPLC-ECD) and gas chromatography with mass spectrometry (GC-MS) detection (for review, see, e.g., Herce et al., 1998 Nitric Oxide 2:324). Artifactual generation of nitrotyrosine during an acid hydrolysis step that precedes such measurements may limit the usefulness of certain GC-MS procedures (see, e.g., Crowley et al., 1998 Anal. Biochem. 259:127). Alternatively, single proteins have been examined for nitrotyrosine content by electrospray mass spectrometry after a nitration step in vitro, such as treatment with peroxynitrite or tetranitromethane. Proteins that have been so analyzed include, for example, superoxide dismutase (Yamakura et al., 1998 J. Biol. Chem. 273:14085), surfactant protein A (Greis et al., 1996 Arch. Biochem. Biophys. 335:396) and non-adenylated glutamine synthetase (Berlett et al., 1998 Proc. Nat. Acad. USA 95:2784). In all of these studies, a characteristic shift of +45 Da in the molecular ion of a specific peptide was observed corresponding to the nitrotyrosine modification. None of the above referenced descriptions of protein nitrotyrosine determination, however, pertain to characterization of a specifically identified tyrosine nitrated protein from a biological source or subject such as a patient sample, nor is the protein tyrosine nitration profile of more than one specific protein contemplated, nor is the amino acid sequence fine specificity of protein tyrosine nitration (e.g., amino acid sequence position of an oxidatively modified tyrosine residue) considered.

Clearly there is a need for improved methods for detecting and monitoring reactive free radical modification of proteins, and in particular protein tyrosine nitration, where such modifications have been implicated in critical cell regulatory mechanisms and in numerous pathological conditions. Particularly useful would be compositions and methods for the identification of specific proteins that undergo tyrosine nitration, identification of protein tyrosine nitration amino acid sequence fine specificity, and determination of protein tyrosine nitration proteomic profiles that define diseases associated with oxidative protein modification as distinguished from normal, control proteomes. The present invention satisfies these needs and offers a number of related advantages.

### SUMMARY OF THE INVENTION

As described herein, there are provided methods and compositions for identifying oxidative protein modifications, including those associated with a number of disease conditions. Accordingly, in one aspect the present invention provides a method for identifying oxidative modification of a protein, comprising generating a mass spectrum of all or a portion of a protein fraction derived from a biological sample, the protein fraction comprising at least one peptide that includes a nitrotyrosine residue, wherein determination of nitrotyrosine in the sample indicates the protein is oxidatively modified. In one embodiment, the invention provides a method for identifying oxidative modification of a protein, comprising comparing (i) a first mass spectrum of all or a portion of a first protein fraction derived from a first biological sample, the first protein fraction comprising at least one peptide that includes a nitrotyrosine residue, to (ii) a second mass spectrum of all or a portion of a second protein fraction derived from a second biological sample, wherein determination of nitrotyrosine in the second protein fraction indicates that a protein therein is oxidatively modified.

In another embodiment, the present invention provides a method for identifying oxidative modification of a protein, comprising contacting all or a portion of a protein fraction derived from a biological sample with at least one proteolytic agent under conditions and for a time sufficient to generate a plurality of peptide fragments derived from the protein fraction, the protein fraction comprising at least one peptide that includes a nitrotyrosine residue; and generating a mass spectrum of one or more of the peptide fragments, wherein determination of nitrotyrosine in at least one of the peptide fragments indicates that a protein in the biological sample is oxidatively modified. In another embodiment, the invention provides a method for determining protein tyrosine nitration in a subject, comprising isolating at least one protein comprising nitrotyrosine from a biological sample derived from a subject; contacting the protein with at least one proteolytic agent under conditions and for a time sufficient to generate a plurality of peptide fragments derived from the protein; and comparing a mass spectrum of one or more of the peptide fragments to a mass spectrum of a control sample containing nitrotyrosine, and therefrom determining protein nitration in the subject. In certain embodiments of any of the above described methods, the mass spectrum is generated by matrix assisted laser desorption ionization mass spectrometry. In a further embodiment, determination of nitrotyrosine comprises detection in the mass spectrum of (a) a peptide comprising nitrotyrosine; (b) a peptide comprising nitrotyrosine that lacks one oxygen atom; and (c) a peptide comprising nitrotyrosine that lacks two oxygen atoms. In certain other embodiments of any of the above described methods, the mass spectrum is generated by matrix assisted laser desorption ionization time-of-flight mass spectrometry, and determination of nitrotyrosine comprises detection in the mass spectrum of (a) a peptide comprising nitrotyrosine; (b) a peptide comprising nitrotyrosine that lacks one oxygen atom; and (c) a peptide comprising nitrotyrosine that lacks two oxygen atoms.

In another embodiment, the invention provides method for identifying oxidative modification of a protein, comprising comparing (a) a first mass spectrum of a first portion of a protein fraction derived from a biological sample, wherein the protein fraction comprises at least one peptide that includes a nitrotyrosine residue, to (b) a second mass spectrum of a second portion of the protein fraction derived from the biological sample, wherein the second mass spectrum is generated (i) subsequent to exposure of the second portion to conditions sufficient to convert nitrotyrosine to aminotyrosine, or (ii) subsequent to contacting the second portion with sodium dithionite under conditions and for a time sufficient to convert nitrotyrosine to aminotyrosine, wherein the second portion of the protein fraction comprises at least one peptide that includes an aminotyrosine residue derived from nitrotyrosine, and wherein determination of nitrotyrosine in the first portion and of amino tyrosine in the second portion indicates that at least one protein in the biological sample is oxidatively modified. In certain further embodiments, prior to the step of comparing, the protein fraction is contacted with at least one proteolytic agent under conditions and for a time sufficient to generate a plurality of peptide fragments derived from the protein fraction. In certain other further embodiments, the peptide that includes an aminotyrosine residue derived from nitrotyrosine undergoes sidechain loss of aminotyrosine.

In another embodiment, the invention provides a method for detecting in a subject the presence of, or risk for having a disease associated with oxidative modification of a protein, comprising generating a mass spectrum of all or a portion of a protein fraction of a biological sample derived from a subject suspected of having or being at risk for having a disease associated with oxidative modification of a protein, the protein fraction comprising at least one peptide that includes a nitrotyrosine residue, wherein determination of nitrotyrosine in the sample indicates the protein is oxidatively modified, and therefrom detecting risk for or presence of a disease in the subject.

In another embodiment, the invention provides a method for detecting in a subject the presence of, or risk for having a disease associated with oxidative modification of a protein, comprising comparing (i) a first mass spectrum of all or a portion of a first protein fraction of a biological sample derived from a first subject suspected of having or being at risk for having a disease associated with oxidative modification of a protein, the first protein fraction comprising at least one peptide that includes a nitrotyrosine residue, to (ii) a second mass spectrum of all or a portion of a second protein fraction of a biological sample derived from a second subject known to be free of a presence or risk for having a disease associated with oxidative modification of a protein, the second protein fraction lacking nitrotyrosine, wherein determination of the presence of nitrotyrosine in the first protein fraction and the absence of nitrotyrosine in the second protein fraction indicates risk for having or presence of a disease in the first subject.

In yet another embodiment, the invention provides a method for identifying a protein that is oxidatively modified in a disease associated with oxidative modification of a protein, comprising comparing (i) a first mass spectrum of all or a portion of a first protein fraction of a biological sample derived from a first subject having or being at risk for having a disease associated with oxidative modification of a protein, the first protein fraction comprising at least one peptide that includes a nitrotyrosine residue, to (ii) a second mass spectrum of all or a portion of a second protein fraction of a biological sample derived from a second subject known to be free of a presence or risk for having a disease associated with oxidative modification of a protein, the second protein fraction lacking nitrotyrosine, wherein determination of the presence of nitrotyrosine in the first protein fraction and the absence of nitrotyrosine in the second protein fraction indicates risk for having or presence of a disease in the first subject; and determining the protein from which the at least one peptide that includes a nitrotyrosine residue is derived, and therefrom identifying a protein that is oxidatively modified in the disease.

In still another embodiment, the present invention provides a method of identifying a suitable agent for treating a disease associated with oxidative modification of a protein, comprising comparing (i) a first mass spectrum of all or a portion of a first protein fraction of a biological sample derived from a subject having or being at risk for having a disease associated with oxidative modification of a protein, prior to contacting the sample with a candidate agent, the first protein fraction comprising at least one peptide that includes a nitrotyrosine residue, to (ii) a second mass spectrum of all or a portion of a second protein fraction of a biological sample derived from the subject subsequent to contacting the sample with the candidate agent, wherein determination of a decreased level of nitrotyrosine in the second mass spectrum relative to the first mass spectrum indicates the agent reduces oxidative protein modification.

According to another embodiment, there is provided a method of identifying a suitable agent for treating a disease associated with oxidative modification of a protein, comprising comparing at least one biological activity of a protein identified according to the method for identifying a protein that is oxidatively modified in a disease associated with oxidative modification of a protein as described above, in the absence of a candidate agent, to the biological activity of the protein in the presence of the candidate agent, wherein an alteration of the activity indicates suitability of the agent for treating a disease associated with oxidative protein modification. In another embodiment, the invention provides a method for identifying oxidative modification of a proteome, comprising generating a mass spectrum of all or a portion of a protein fraction derived from a biological sample, the protein fraction comprising a plurality of proteins that each contain a nitrotyrosine residue, wherein determination of nitrotyrosine in the sample indicates the proteins are oxidatively modified.

These and other aspects of the present invention will become evident upon reference to the following detailed description and attached drawings. In addition, various references are set forth below which describe in more detail certain procedures or compositions and are therefore incorporated by reference in their entireties.

### BRIEF DESCRIPTION OF THE DRAWINGS

FIG. 1 shows an example of a representative overall scheme for nitrotyrosine identification in a protein or mixture of proteins by mass spectrometry and proteomics.

FIG. 2 shows linear positive-ion MALDI spectra of BSA and nitrated BSA (N-BSA). The measured singly charged molecular ions were m/z 66,438 for BSA (vs. m/z 66431, calculated) and m/z 66,857 for N-BSA (ΔM=419 Da).

FIG. 3 shows linear positive-ion MALDI-TOF spectra of unseparated tryptic digests of BSA (FIG. 3A) and of nitrated BSA generated following treatment of BSA with tetranitromethane (FIG. 3B). The (M+H)⁺ ions at m/z 927.4 and 1479.8 were significantly reduced in abundance in the nitrated BSA digest, as shown in the lower spectrum (FIG. 3B), which included three new ions (denoted with *) at m/z 972.5.1, 1484 and 1524.6, corresponding to the nitration (addition of 45 Da) of tyrosine in each peptide. Associated with each of these three new molecular ions were ions that were reduced in mass by 16 Da (m/z 956.5, and 1468.5 and 1508.6; ●) and by 32 Da (m/z 940.5, 1452.4 and 1492.8; ∘), respectively, as described in greater detail below.

FIG. 4 shows the molecular ion region of a MALDI-TOF spectrum of synthetic peptide AAFGY(NO₂)AR taken in the linear mode (FIG. 4A) and reflectron (FIG. 4B) mode. The structures of 3-nitrotyrosine and of photodecomposition products (according to non-limiting theory as described below) are shown next to the various ions. Several small ions labeled with asterisks (FIG. 4B) correspond to theorized metastable peaks. Also according to non-limiting theory, slight increases in the abundance of the ion at m/z 769.4 over what would be expected for the C-13 isotope peak for the amino-tyrosine products (m/z 770.4) in both spectra suggested the formation of a catechol product.

FIG. 5 shows a MALDI-PSD spectrum of BSA tryptic peptide Y(NO₂)LYEIAR with timed ion selection of the lowest mass photodecomposition components (M+H−30)⁺ and (M+H−32)⁺ at m/z 942.4 and 940.4.

FIG. 6 shows tandem MALDI-Q-TOF spectra of synthetic peptide AAFGY(NO₂)AR with the MH⁺ ion at m/z 800.4 selected for collisional activation. Boxed inset (top right) shows the molecular ion region of a normal MALDI-MS scan from which the precursor ion at m/z 800.4 was selected for the subsequent MS/MS experiment. The −16 (m/z 784) Da and −32 (m/z 768) Da photodecomposition fragments were absent in the resulting MS/MS spectra. Inset on top left shows expanded region (×10) of the MS/MS spectrum containing the nitrotyrosine immonium ion at m/z 181.

FIG. 7 shows MALDI-TOF spectra with post-source decay (PSD) of synthetic peptide AAFGY(NO₂)AR. Timed ion selection was set for transmission of precursor ions at (FIG. 7A) m/z 800.4 Da and (FIG. 7B) 768.4 Da and 770.4.

FIG. 8 shows comparative immonium ion regions for the precursor (parent) ions at (FIG. 8A) m/z 800, (FIG. 8B) m/z 786 and 784 (with m/z 880, 770 and 768), and (FIG. 8C) m/z 770 and 768 for the peptide AAFGY(NO₂)AR. Structures of nitrotyrosine and corresponding photochemical decomposition product immonium ions are shown next to the corresponding masses.

FIG. 9 shows changes in the relative ion abundances of the molecular ion (m/z 800.4) and photo-decomposition products (m/z 786.3, 784.3, 770.3 and 768.3) for peptide AAFGY(NO₂)AR under linear MALDI-MS conditions at different loading amounts (concentrations). Amounts of sample spotted were (FIG. 9A) 2.5 nmole, (FIG. 9B) 0.25 nmole, and (FIG. 9C) 2.5 pmole.

FIG. 10 shows MALDI-TOF with PSD of reduced 3-nitrotyrosine peptide AAFGY(NH₂)AR. The abundant ion at m/z 678 (−Y*) corresponded to the loss of the aminotyrosine side chain.

### DETAILED DESCRIPTION OF THE INVENTION

According to the present invention, there are provided compositions and methods for the identification of nitrotyrosine modifications at the sequence level in a single targeted protein or in a complex mixtures of proteins. The invention thus relates in pertinent part to the unique chemical and photochemical properties of nitrotyrosine residues in peptides and proteins, in conjunction with standard immunochemical methods, modern spectrometry and protein bioinformatics software tools to identify peptides and proteins that contain this modification. Determining the pattern of nitrotyrosine modifications at the peptide and/or protein level in a complex protein mixture obtained from a biological sample as provided herein (i.e., at the proteomic level) provides, in certain embodiments, diagnostic information that could aid in the identification of specific disease states. In certain other embodiments the invention provides methods for evaluating the effects of candidate therapeutic agents (e.g., drugs) on the protein tyrosine oxidative process. Thus, in certain embodiments described in greater detail below, such candidate agents may cause one or more specific alterations (e.g., increases or decreases in a statistically significant manner) in the overall pattern of nitrotyrosine formation, preferably in some beneficial fashion.

As described herein, the profiling of nitrotyrosine modifications in a preparation containing one or a plurality of proteins and/or peptides from a biological sample may be referred to as the “proteomics” of nitrotyrosine modification, and provides a powerful technology for, inter alia, diagnosing diseases associated with protein tyrosine oxidative modification (e.g., degenerative diseases), identifying new protein candidates that may be important therapeutic targets in the protein tyrosine oxidative process, and screening candidate agents in assays to identify and/or evaluate therapeutic drugs for diseases associated with protein tyrosine oxidative modification.

The present invention is directed in part to the unexpected observation that under certain mass spectrometric conditions, 3-nitrotyrosine generates a unique and readily detectable signature profile that provides a highly selective and sensitive method for the analysis and characterization of nitrotyrosine-containing peptides. As described herein, procedures are thus provided for monitoring oxidative damage at the level of proteins or peptides derived therefrom. The invention also relates in part to identification of protein oxidation phenotypes at the proteomic level (i.e., a profile at the level of all detectable expressed proteins in a biological sample or protein fraction thereof) based on the determination of 3-nitrotyrosine in specific protein members of a proteome, and in certain further embodiments, on the determination of 3-nitrotyrosine residues situated at specific positions within such proteins. Without wishing to be bound by theory, and according to certain of these embodiments, peroxynitrite radicals that result from oxidative stress and that mediate protein tyrosine nitration may do so by a non-random and specific process, which defines a regulated mechanism for posttranslational protein modification.

In brief, and as described in greater detail below, according to the present invention a biological sample is obtained from a subject or biological source, and from such a sample a protein fraction is prepared. Depending on a variety of factors (including, e.g., the biological source, the condition of the source with regard to oxidative phenotype, the type of preparation of the protein fraction, etc.), and in preferred embodiments, the protein fraction comprises at least one protein or peptide that includes a nitrotyrosine residue. In particularly preferred embodiments, the protein fraction comprises a plurality of proteins and/or peptides, each of which includes at least one nitrotyrosine residue. The protein fraction may be treated with a proteolytic agent under conditions and for a time sufficient to generate a plurality of peptide fragments, which may then be analyzed for the presence of nitrotyrosine by mass spectrometry (MS). Peptides in which nitrotyrosine is detected as provided herein are then characterized on the basis of mass and/or amino acid sequence properties. Comparison of peptide sequences so identified as containing oxidatively modified tyrosine (e.g., nitrotyrosine) to known protein and peptide sequences (e.g., by searching protein sequence databases) permits determination of the identity or identities of the protein(s) and/or peptides that have been oxidatively modified in the subject or biological source.

Thus, the present invention is directed in pertinent part to the use of mass spectrometry, and in particular to the use of matrix assisted laser desorption ionization time-of-flight (MALDI-TOF) mass spectrometry, for the analysis of peptides containing nitrotyrosine as obtained from a subject or biological source as provided herein. The instant disclosure provides the surprising observation that tyrosine nitrated peptides generate a unique signature MS spectrum triplet in MALDI-TOF, comprising detection in the mass spectrum of (i) a peptide comprising nitrotyrosine, (ii) a peptide comprising nitrotyrosine that lacks one oxygen atom, and (iii) a peptide comprising nitrotyrosine that lacks two oxygen atoms. Without wishing to be bound by theory, according to the present invention, nitrotyrosine-containing peptides that lack one or two oxygen atoms may be photochemical reaction products of nitrotyrosine-containing peptides that are generated during exposure of such peptides to the laser employed in MALDI (e.g., N₂ laser, λ=337 nm; Nd:YAG laser, λ=355 nm; other lasers in the UV spectral region such as HeNe and Ar lasers would also be expected to promote photo-decomposition of nitrotyrosine).

In certain embodiments, the present invention relates in pertinent part to the unexpected observation that identification of a nitrotyrosine-containing peptide by MALDI-TOF based on the unique MS signature triplet as just described, can be confirmed by subjecting an aliquot containing such a nitrotyrosine-containing peptide to mild reducing conditions that promote quantitative conversion of nitrotyrosine to aminotyrosine without undesirable side reactions that alter other constituents of the peptide, followed by MS characterization of the resulting derivative peptide. For example, peptides containing 3-nitrotyrosine may be quantitatively converted by exposure to sodium dithionite (Na₂S₂O₄; sodium hydrosulfite) into a single 3-aminotyrosine molecular ion peak with higher relative abundance than, and exhibiting a mass shift to a position 30 daltons less than, the major nitrotyrosine peak detected following MALDI. Other reducing agents known to the art may also be useful to effect conversion of nitrotyrosine to aminotyrosine, and selection of such agents (e.g., dithiothreitol, dithioerythritol, 2-mercaptoethanol, sodium borohydride and the like) and conditions for their use can be performed readily and without undue experimentation based on the disclosure provided herein.

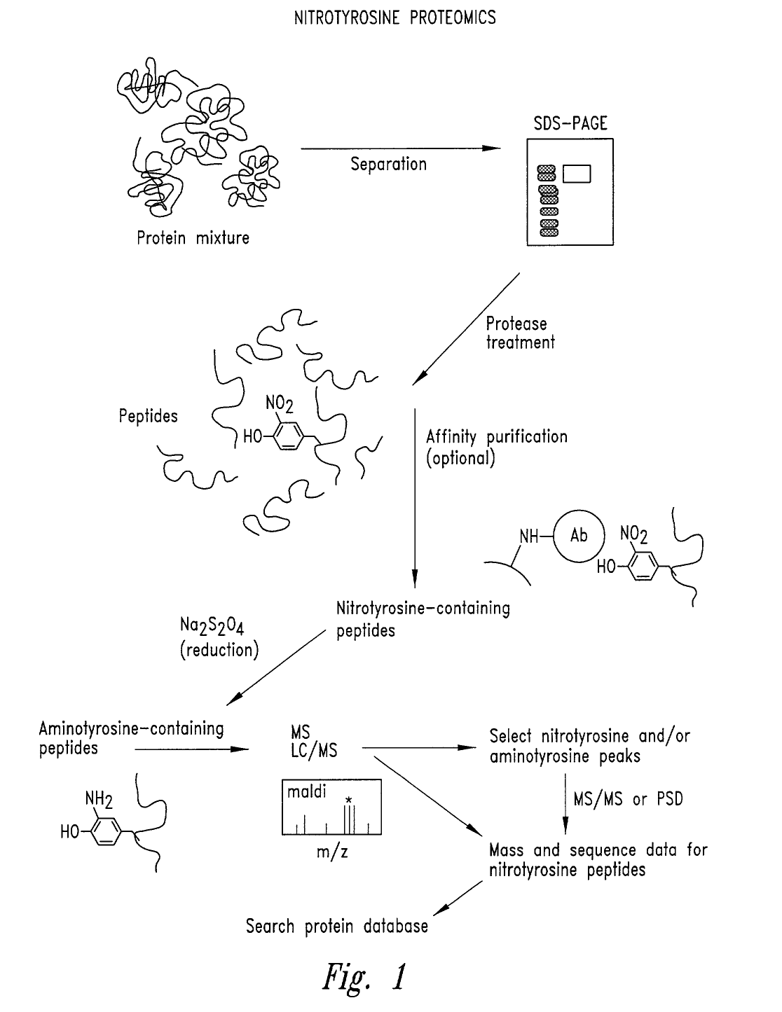

Similarly, because certain laser light sources (e.g., infrared or IR lasers, Cramer et al., 1998 Anal. Chem. 70, 4939-44) would not be expected to promote photochemical conversion of nitrotyrosine to aminotyrosine as described above, related embodiments may be directed to a comparison of the UV and IR MALDI-TOF mass spectra of a tyrosine nitrated peptide, wherein the UV MALDI spectra exhibit the signature triplet while the IR MALDI spectra exhibit a major nitrotyrosine peak. Thus, according to the present invention, a person having ordinary skill in the art can readily profile a proteome (or a fraction or portion thereof) by identifying nitrotyrosine-containing peptides therein, using a rapid and sensitive technique that is operative even where such peptides are present as components of a complex peptide mixture. In certain other preferred embodiments, the protein fraction derived from the biological sample is a positively selected protein fraction that has been immunoaffinity isolated using an antibody specific for nitrotyrosine (FIG. 1). In certain other preferred embodiments, the protein fraction derived from the biological sample (e.g., unselected or immunoaffinity isolated) is optionally further fractionated prior to the generation of peptide fragments using a proteolytic agent. In certain other preferred embodiments, nitrotyrosine-containing peptides are optionally isolated from the plurality of peptide fragments generated following contact of the protein fraction with one or more proteolytic agents, by immunoaffinity selection using an immobilized antibody specific for nitrotyrosine (FIG. 1). Optionally and in certain embodiments, peptide fragments are separated and/or analyzed by liquid chromatography (LC) followed by MS, and peaks are characterized according to MS/MS or post-source decay (PSD) methodologies with which those having ordinary skill in the art will be familiar based on the disclosure herein (see, e.g., Matsui et al., 1997 Electrophoresis 18:409; Shevchenko et al., 1996 Anal. Chem. 68:850; Biemann, K. et al., 1987 Mass. Spectrom. Revs. 6, 1-77; Gillece, C. B. et al., 1996 Methods Enzymol 271, 427-48; Chaurand et al., 1999 J. Am. Soc. Mass Spectrom. 10, 91-103; Courchesne et al., 1999 Methods Mol. Biol. 112, 487-511; Dancik et al., 1999 J. Comput. Biol. 6, 327-42; Jensen et al., 1999 Methods Mol. Biol. 112, 571-88; Shevchenko et al., 1997 J. Protein Chem. 16, 481-90.)

Biological samples may comprise any tissue or cell preparation in which at least one protein can be detected, including a tyrosine-containing protein having one or more tyrosine residues that may undergo oxidative modification, and may vary in nature accordingly, depending on the particular protein(s) to be compared. Biological samples may be provided by obtaining a blood sample, biopsy specimen, tissue explant, organ culture or any other tissue or cell preparation from a subject or a biological source. The subject or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid or cytoplasmic hybrid “cybrid” cell lines, differentiated or differentiatable cell lines, transformed cell lines and the like. In certain preferred embodiments of the invention, the subject or biological source may be suspected of having or being at risk for having a disease associated with oxidative modification of one or more proteins, and in certain preferred embodiments of the invention the subject or biological source may be known to be free of a risk or presence of such a disease.

In certain aspects of the invention, biological samples comprising a protein fraction containing at least one peptide that includes a nitrotyrosine residue may be obtained from the subject or biological source before and after contacting the subject or biological source with a candidate agent, for example to identify a candidate agent capable of effecting a change in the level of nitrotyrosine as provided herein, relative to the level before exposure of the subject or biological source to the agent.

In a most preferred embodiment of the invention, the biological sample comprising a protein fraction containing at least one nitrotyrosine residue may comprise whole blood, and may in another preferred embodiment comprise a crude buffy coat fraction of whole blood, which is known in the art to comprise further a particulate fraction of whole blood enriched in white blood cells and platelets and substantially depleted of erythrocytes. Those familiar with the art will know how to prepare such a buffy coat fraction, which may be prepared by differential density sedimentation of blood components under defined conditions, including the use of density dependent separation media, or by other methods. In other preferred embodiments, the biological sample comprising a protein fraction containing at least one nitrotyrosine residue may comprise an enriched, isolated or purified blood cell subpopulation fraction such as, for example, lymphocytes, polymorphonuclear leukocytes, granulocytes and the like. Methods for the selective preparation of particular hematopoietic cell subpopulations are well known in the art (see, e.g., Current Protocols in Immunology, J. E. Coligan et al., (Eds.) 1998 John Wiley & Sons, NY).

According to certain embodiments of the invention, the particular cell type or tissue type from which a biological sample is obtained may influence qualitative or quantitative aspects of at least one protein or peptide that includes a nitrotyrosine residue contained therein, relative to the corresponding protein fraction comprising proteins and/or peptides obtained from distinct cell or tissue types of a common biological source. It is therefore within the contemplation of the invention to quantify at least one species of protein or peptide in biological samples from different cell or tissue types as may render the advantages of the invention most useful for a particular disease associated with oxidative protein tyrosine nitration, and further for a particular degree of progression of such disease. The relevant cell or tissue types will be known to those familiar with such diseases.

In particularly preferred embodiments of the present invention, a protein fraction is derived from the biological sample as provided herein. A protein fraction may be any preparation that contains at least one protein that is present in the sample (preferably a protein having at least one tyrosine residue that may undergo oxidative modification to nitrotyrosine) and which may be obtained by processing a biological sample according to any biological and/or biochemical methods useful for isolating or otherwise separating a protein from its biological source. Those familiar with the art will be able to select an appropriate method depending on the biological starting material and other factors. Such methods may include, but need not be limited to, cell fractionation, density sedimentation, differential extraction, salt precipitation, ultrafiltration, gel filtration, ion-exchange chromatography, partition chromatography, hydrophobic chromatography, reversed-phase chromatography, one- and two-dimensional electrophoresis, affinity techniques or any other suitable separation method.

Affinity techniques are particularly useful in the context of the present invention, and may include any method that exploits a specific binding interaction with a nitrotyrosine-containing protein or peptide to effect a separation. For example, an affinity technique such as binding of a nitrotyrosine-containing protein or peptide to an immobilized nitrotyrosine-specific antibody may be a particularly useful affinity technique. Other useful affinity techniques include immunological techniques for isolating specific proteins or peptides, which techniques rely on specific binding interaction between antibody combining sites for antigen and antigenic determinants present in the proteins or peptides. Immunological techniques include, but need not be limited to, immunoaffinity chromatography, immunoprecipitation, solid phase immunoadsorption or other immunoaffinity methods. See, for example, Scopes, R. K., Protein Purification: Principles and Practice, 1987, Springer-Verlag, NY; Weir, D. M., Handbook of Experimental Immunology, 1986, Blackwell Scientific, Boston; Deutscher, M. P., Guide to Protein Purification, 1990, Methods in Enzymology Vol. 182, Academic Press, New York; and Hermanson, G. T. et al., Immobilized Affinity Ligand Techniques, 1992, Academic Press, Inc., California; which are hereby incorporated by reference in their entireties, for details regarding techniques for isolating and characterizing proteins and peptides, including affinity techniques.

The term “isolated” means that the material is removed from its original environment (e.g., the natural environment if it is naturally occurring). For instance, a naturally occurring protein or peptide present in a living animal is not isolated, but the same protein or peptide, separated from some or all of the co-existing materials in the natural system, is isolated. Thus, for example, such proteins could be part of a multisubunit complex or a membrane vesicle, and/or such peptides could be part of a composition, and still be isolated in that such complex, vesicle or composition is not part of its natural environment.

“Biological activity” of a protein may be any detectable parameter that directly relates to a condition, process, pathway, dynamic structure, state or other activity involving the protein and that permits detection of altered protein function in a biological sample from a subject or biological source, or in a preparation of the protein isolated therefrom. The methods of the present invention thus pertain in part to such correlation where the protein having biological activity may be, for example, an enzyme, a structural protein, a receptor, a ligand, a membrane channel, a regulatory protein, a subunit, a complex component, a chaperone protein, a binding protein or a protein having a biological activity according to other criteria including those provided herein.

“Altered biological activity” of a protein may refer to any condition or state, including those that accompany a disease associated with oxidative modification of a protein, where any structure or activity that is directly or indirectly related to a particular protein's function (or multiple functions) has been changed in a statistically significant manner relative to a control or standard. Altered biological activity may have its origin in oxidatively modified structures or oxidative events as well as in oxidation-independent structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like. According to certain embodiments as provided herein, altered biological activity of a protein may also result from direct or indirect interaction of a biologically active protein with an introduced agent such as an agent for treating a disease associated with oxidative modification of proteins as described herein, for example, a small molecule.

Additionally, altered biological activity of a protein may result in altered respiratory, metabolic or other biochemical or biophysical activity in some or all cells of a biological source. As non-limiting examples, markedly impaired ETC activity may be related to altered biological activity of at least one protein, as may be generation of increased free radicals such as reactive oxygen species (ROS) or defective oxidative phosphorylation. As further examples, altered mitochondrial membrane potential, induction of apoptotic pathways and formation of a typical chemical and biochemical crosslinked species within a cell, whether by enzymatic or non-enzymatic mechanisms, may all be regarded as indicative of altered protein biological activity. These and other non-limiting examples of altered protein biological activity are described in greater detail below.

In particularly preferred embodiments of the present invention, all or a portion of a protein fraction derived from a biological sample as provided herein may be contacted with one or more proteolytic agents under conditions and for a time sufficient to generate a plurality of peptide fragments derived from the protein fraction. Peptide fragments are typically continuous portions of a polypeptide chain derived from a protein of the protein fraction, which portions may be up to about 100 amino acids in length, preferably up to about 50 amino acids in length, more preferably up to about 30 amino acids in length, and still more preferably up to about 15-20 amino acids in length. In particularly preferred embodiments peptide fragments are 10-15 amino acids in length, and in other preferred embodiments peptide fragments may be 2-12 amino acids long.

A variety of proteolytic agents and suitable conditions for using them are known in the art, any of which may be useful according to certain embodiments of the present invention wherein peptide fragments are generated. Particularly preferred are proteolytic agents that are proteolytic enzymes or proteases, for example trypsin, Glu-C protease (Staphylococcal V8 protease), Lys-C protease, Arg-C protease, or other proteases known in the art to cleave peptides at specific amino acid linkages, typically at a relatively limited number of cleavage sites within a protein or polypeptide. Other useful proteolytic agents that are proteolytic enzymes include serine proteases, for example, chymotrypsin, elastase and trypsin; thiol proteases, such as papain or yeast proteinase B; acid proteases, including, e.g., pepsin or cathepsin D; metalloproteinases (e.g., collagenases, microbial neutral proteinases); carboxypeptidases; N-terminal peptidases or any other proteolytic enzymes that those having ordinary skill in the art will recognize may be employed to generate peptide fragments as provided herein (see, e.g., Bell, J. E. and Bell, E. T., Proteins and Enzymes, 1988 Prentice-Hall, Englewood Cliffs, N.J.; Worthington Enzyme Manual, V. Worthington, ed., 1993 Worthington Biochemical Corp., Freehold, N.J.).

Alternatively, in certain embodiments it may be desirable to use proteolytic agents that are chemical agents, for example HCl, CNBr, formic acid, N-bromosuccinimide, BNPS-skatole, o-iodosobenzoic acid/p-cresol, Cyssor, 2-nitro-5-thiocyanobenzoic acid, hydroxylamine, pyridine/acetic acid or other chemical cleavage procedures (see, e.g., Bell and Bell, 1988, and references cited therein).

As noted above, oxidative damage to proteins, such as protein modification that results from reactive free radical activity in biological systems, is an underlying feature in the pathogenesis of a number of diseases. Accordingly, a “disease associated with oxidative modification of a protein” may include any disease in which at least one protein or peptide is oxidatively (e.g., covalently) and, in most cases, inappropriately modified. In highly preferred embodiments, at least one protein or peptide in a subject or biological source having a disease associated with oxidative modification of a protein includes a nitrated tyrosine residue as a result of disease-associated oxidative damage. Thus, such a disease may have a basis in a respiratory or metabolic or other defect, whether mitochondrial or extramitochondrial in origin. Diseases associated with oxidative modification of proteins may include Alzheimer's disease (AD), diabetes mellitus, Parkinson's disease, amyotrophic lateral sclerosis (ALS), atherosclerosis and other degenerative and inflammatory diseases. Those familiar with the art will be aware of clinical criteria for diagnosing certain of these diseases, which diagnostic criteria are augmented in view of the subject invention methods and compositions.

In order to determine whether a mitochondrial component may contribute to a particular disease associated with oxidative modification of a protein, it may be useful to construct a model system for diagnostic tests and for screening candidate therapeutic agents in which the nuclear genetic background may be held constant while the mitochondrial genome is modified. It is known in the art to deplete mitochondrial DNA from cultured cells to produce ρ⁰ cells, thereby preventing expression and replication of mitochondrial genes and inactivating mitochondrial function. It is further known in the art to repopulate such ρ⁰ cells with mitochondria derived from foreign cells in order to assess the contribution of the donor mitochondrial genotype to the respiratory phenotype of the recipient cells. Such cytoplasmic hybrid cells, containing genomic and mitochondrial DNAs of differing biological origins, are known as cybrids. See, for example, International Publication Number WO 95/26973 and U.S. Pat. No. 5,888,498 which are hereby incorporated by reference in their entireties, and references cited therein.

According to the present invention, a level of at least one protein or peptide containing nitrotyrosine is determined in a biological sample from a subject or biological source. For subjects that are asymptomatic, that exhibit a pre-disease phenotype or that meet clinical criteria for having or being at risk for having a particular disease, such determination may have prognostic and/or diagnostic usefulness. For example, where other clinical indicators of a given disease are known, levels of at least one protein or peptide containing nitrotyrosine in subjects known to be free of a risk or presence of such disease based on the absence of these indicators may be determined to establish a control range for such level(s). The levels may also be determined in biological samples obtained from subjects suspected of having or being at risk for having the disease, and compared to the control range determined in disease free subjects. Those having familiarity with the art will appreciate that there may be any number of variations on the particular subjects, biological sources and bases for comparing levels of at least protein or peptide containing nitrotyrosine that are useful beyond those that are expressly presented herein, and these additional uses are within the scope and spirit of the invention.

For instance, determination of levels of at least one protein or peptide containing nitrotyrosine may take the form of a prognostic or a diagnostic assay performed on a skeletal muscle biopsy, on whole blood collected from a subject by routine venous blood draw, on buffy coat cells prepared from blood or on biological samples that are other cells, organs or tissue from a subject. Alternatively, in certain situations it may be desirable to construct cybrid cell lines using mitochondria from either control subjects or subjects suspected of being at risk for a particular disease associated with oxidative modification of proteins. Such cybrids may be used to determine levels of at least one peptide or protein containing nitrotyrosine for diagnostic or predictive purposes, or as biological sources for screening assays to identify agents that may be suitable for treating the disease based on their ability to alter (e.g., to increase or decrease in a statistically significant manner) the levels of at least one protein or peptide containing nitrotyrosine in treated cells.

In one embodiment of this aspect of the invention, therapeutic agents or combinations of agents that are tailored to effectively treat an individual patient's particular disease may be identified by routine screening of candidate agents on cybrid cells constructed with the patient's mitochondria. In another embodiment, a method for identifying subtypes of the particular disease is provided, for example, based on differential effects of individual candidate agents on cybrid cells constructed using mitochondria from different subjects diagnosed with the same disease.

As noted above, in certain preferred embodiments of the present invention there is provided a method for identifying oxidative modification of a protein comprising generating a mass spectrum of a protein fraction or peptide fragment comprising a nitrotyrosine residue, wherein the mass spectrum is preferably generated using MALDI-TOF. By way of background, in 1987, matrix-assisted laser desorption/ionization mass spectrometry (MALDI) was introduced by Hillenkamp and Karas, and since has become a very powerful bioanalytical tool (Anal. Chem. 60:2288-2301, 1988; see also Burlingame et al., Anal. Chem. 68:599-651, 1996 and references cited therein). The success of MALDI in the area of protein science can be attributed to several factors. The greatest of these is that MALDI can be rapidly applied as an analytical technique to analyze small quantities of virtually any protein (practical sensitivities of ˜1 pmole protein loaded into the mass spectrometer). The technique is also extremely accurate. Beavis and Chait demonstrated that the molecular weights of peptides and proteins can be determined to within ˜0.01% by using methods in which internal mass calibrants (x-axis calibration) are introduced into the analysis (Anal. Chem. 62:1836-40, 1990). MALDI can also be made quantitative using a similar method in which internal reference standards are introduced into the analysis for ion signal normalization (y-axis calibration). Quantitative determination of proteins and peptides is possible using this approach with accuracies on the order of ˜10% (Nelson et al., Anal. Chem. 66:1408-15, 1994). Finally, MALDI is extremely tolerant of large molar excesses of buffer salts and, more importantly, the presence of other proteins.

With the high tolerance towards buffer salts and other biomolecular components comes the ability to directly analyze complex biological mixtures. Many examples exist where MALDI is used to directly analyze the results of proteolytic or chemical digestion of polypeptides (see Burlingame et al., supra). Other examples extend to elucidating post-translational modifications (namely carbohydrate type and content), a process requiring the simultaneous analysis of components present in a heterogeneous glycoprotein mixture (Sutton et al., Techniques in Protein Chemistry III, Angeletti, Ed., Academic Press, Inc., New York, pp. 109-116, 1993). Arguably, the most impressive use of direct mixture analysis is the screening of natural biological fluids. In that application, proteins are identified, as prepared directly from the host fluid, by detection at precise and characteristic mass-to-charge (m/z) values (Tempst et al., Mass Spectrometry in the Biological Sciences, Burlingame and Carr, Ed., Humana Press, Totowa, N.J., p.105, 1996).

The use of an affinity ligand-derivatized support to selectively retrieve a target analyte specifically for MALDI analysis was first demonstrated by Hutchens and Yip (Rapid Commun. Mass Spectrom. 7:576-80, 1993). Those investigators used single-stranded DNA-derivatized agarose beads to selectively retrieve a protein, lactoferrin, from pre-term infant urine by incubating the beads with urine. The agarose beads were then treated as the MALDI analyte—a process involving mixing with a solution-phase MALDI matrix followed by deposition of the mixture on a mass spectrometer probe. MALDI then proceeded in the usual manner. Results indicated that the derivatized beads selectively retrieved and concentrated the lactoferrin; enough so to enable ion signal in the MALDI mass spectrum adequate to unambiguously identify the analyte at the appropriate m/z value (81,000 Da). A number of variations on this approach have since been reported. These include the use of immunoaffinity precipitation for the MALDI analysis of transferrins in serum (Nakanishi et al., Biol. Mass Spectrom. 23:230-33, 1994), screening of ascites for the production of monoclonal antibodies (Papac et al., Anal. Chem. 66:2609-13, 1994), and the identification of linear epitope regions within an antigen (Zhao et al., Anal. Chem. 66:3723-26, 1994). Even more recently, the affinity capture approaches have been made rigorously quantitative by incorporating mass-shifted variants of the analyte into the analysis (Nelson et al. Anal. Chem. 67:1153-58, 1995). The variants are retained throughout the analysis (in the same manner as the true analyte) and observed as unique (resolved) signals in the MALDI mass spectrum. Quantification of the analyte is performed by equating the relative ion signals of the analyte and variant to an analyte concentration.

Suitable mass spectrometers include, but are not limited to, a magnetic sector mass spectrometer, a Fourier transform ion cyclotron resonance (FTICR) mass spectrometer, a quadrapole (rods or ion trap) mass spectrometer and a time-of-flight (TOF) mass spectrometer. In a preferred embodiment, the mass spectrometer is a time TOF mass spectrometer.

Since large molecules, such as peptides and proteins, are generally too large to be desorbed/ionized intact, a matrix is used to assist laser desorption/ionization of the same. This technique is referred to as matrix assisted laser desorption/ionization or (MALDI), and the matrix agent is referred to as a “MALDI matrix.” In short, the analyte is contacted with a suitable MALDI matrix and allowed to crystallize. Suitable MALDI matrix materials are known to those skilled in this field, and include, for example, derivatives of cinnamic acid such as α-cyano-4-hydroxycinnamic acid (ACCA) and sinapinic acid (SA).

A first criterion to performing mass spectrometry on the analyte captured by the interactive surface is the generation of vapor-phase ions. In the practice of this invention, such species are generated by desorption/ionization techniques. Suitable techniques include desorption/ionization methods derived from impact of particles with the sample. These methods include fast atom bombardment (FAB—impact of neutrals with a sample suspended in a volatile matrix), secondary ion mass spectrometry (SIMS—impact of keV primary ions generating secondary ions from a surface), liquid SIMS (LSIMS—like FAB except the primary species is an ion), plasma desorption mass spectrometry (like SIMS except using MeV primary ions), massive cluster impact (MCI—like SIMS using large cluster primary ions), laser desorption/ionization (LDI—laser light is used to desorb/ionize species from a surface), and matrix-assisted laser desorption/ionization (MALDI—like LDI except the species are desorbed/ionized from a matrix capable of assisting in the desorption and ionization events). Any of the aforementioned desorption/ionization techniques may be employed in the practice of the present invention. In a preferred embodiment, LDI is employed, and in a more preferred embodiment, MALDI is utilized. For matrix assisted laser desorption ionization/time of flight (MALDI-TOF) analysis or other MS techniques known to those skilled in the art, see, for example, U.S. Pat. Nos. 5,622,824, 5,605,798 and 5,547,835.

In certain aspects, the present invention provides a method of identifying a suitable agent for treating a disease associated with oxidative modification of a protein, comprising comparing (i) a first mass spectrum of all or a portion of a first protein fraction of a biological sample derived from a subject having or being at risk for having a disease associated with oxidative modification of a protein, prior to contacting the sample with a candidate agent, the first protein fraction comprising at least one peptide that includes a nitrotyrosine residue, to (ii) a second mass spectrum of all or a portion of a second protein fraction of a biological sample derived from the subject subsequent to contacting the sample with the candidate agent, wherein determination of a decreased level of nitrotyrosine in the second mass spectrum relative to the first mass spectrum indicates the agent reduces oxidative protein modification.

Candidate agents for use in these and related methods of screening for a modulator of protein or peptide nitrotyrosine according to the present invention may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be administered to a plurality of samples, and then assayed for their ability to increase or decrease the level of at least one indicator of altered mitochondrial function.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666, which are hereby incorporated by reference in their entireties) or other compositions that may include small molecules as provided herein (see e.g., PCT/US94/08542, EP 0774464, U.S. Pat. No. 5,798,035, U.S. Pat. No. 5,789,172, U.S. Pat. No. 5,751,629, which are hereby incorporated by reference in their entireties). Those having ordinary skill in the art will appreciate that a diverse assortment of such libraries may be prepared according to established procedures, and tested for their influence on an indicator of altered mitochondrial function, according to the present disclosure.

The present invention provides compositions and methods that are useful in pharmacogenomics, for the classification and/or stratification of a subject or patient population. In one embodiment, for example, such stratification may be achieved by identification in a subject or patient population of one or more distinct profiles of at least one protein or peptide that contains nitrotyrosine that correlates with a particular disease associated with oxidative modification of proteins. Such profiles may define parameters indicative of a subject's predisposition to develop the particular disease, and may further be useful in the identification of novel subtypes of that disease. In another embodiment, correlation of one or more traits in a subject with at least one protein or peptide that contains nitrotyrosine may be used to gauge the subject's responsiveness to, or the efficacy of, a particular therapeutic treatment. Similarly, where levels of at least one indicator protein or peptide containing nitrotyrosine and risk for a particular disease associated with oxidative modification of proteins are correlated, the present invention provides advantageous methods for identifying agents suitable for treating such disease(s), where such agents affect levels of at least one protein or peptide containing nitrotyrosine in a biological source. Such suitable agents will be those that alter (e.g., increase or decrease) the level of nitrotyrosine in a statistically significant manner. In certain preferred embodiments, a suitable agent alters a nitrotyrosine level in at least one protein or peptide in a manner that confers a clinical benefit, and in certain other, non-exclusive preferred embodiments, a suitable agent alters a nitrotyrosine level by causing it to return to a level detected in control or normal (e.g., disease-free) subjects.

As described herein, determination of levels of at least one protein or peptide that includes a nitrotyrosine residue may also be used to stratify a patient population (i.e., a population classified as having one or more diseases associated with oxidative modification of a protein). Accordingly, in another preferred embodiment of the invention, determination of levels of nitrotyrosine in at least one protein or peptide in a biological sample from an oxidatively stressed subject may provide a useful correlative indicator for that subject. A subject so classified on the basis of nitrotyrosine levels may be monitored using any known clinical parameters for a specific disease referred to above, such that correlation between levels of at least one protein or peptide containing nitrotyrosine and any particular clinical score used to evaluate a particular disease may be monitored. For example, stratification of an AD patient population according to levels of at least one protein or peptide containing nitrotyrosine may provide a useful marker with which to correlate the efficacy of any candidate therapeutic agent being used in AD subjects.

In certain other embodiments, the invention provides a method of treating a patient having a disease associated with oxidative modification of a protein by administering to the patient an agent that substantially restores at least one protein or peptide containing nitrotyrosine to a level found in control or normal subjects (which in some cases may be an undetectable level). In a most preferred embodiment, an agent that substantially restores (e.g., increases or decreases) at least one protein or peptide containing nitrotyrosine to a normal level effects the return of the level of that indicator to a level found in control subjects. In another preferred embodiment, the agent that substantially restores such an indicator confers a clinically beneficial effect on the subject. In another embodiment, the agent that substantially restores the indicator promotes a statistically significant change in the level of at least one protein or peptide containing nitrotyrosine. As noted herein, those having ordinary skill in the art can readily determine whether a change in the level of a particular nitrotyrosine-containing protein or peptide brings that level closer to a normal value and/or clinically benefits the subject, based on the present disclosure. Thus, an agent that substantially restores at least one protein or peptide containing nitrotyrosine to a normal level may include an agent capable of fully or partially restoring such level. These and related advantages will be appreciated by those familiar with the art.

Any of the agents for treating a disease associated with oxidative modification of a protein, identified as described herein, are preferably part of a pharmaceutical composition when used in the methods of the present invention. The pharmaceutical composition will include at least one of a pharmaceutically acceptable carrier, diluent or excipient, in addition to one or more agents for treating a disease associated with oxidative modification of a protein, and, optionally, other components.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

“Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention.

The pharmaceutical compositions that contain one or more agents for treating a disease associated with oxidative modification of a protein may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, intrathecal and intranasal. The term parenteral as used herein includes subcutaneous injections, intravenous, intramuscular, intrasternal, intracavernous, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain, in addition to one or more agents for treating a disease associated with oxidative modification of a protein, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of agent(s) for treating a disease associated with oxidative modification of a protein such that a suitable dosage will be obtained. Typically, this amount is at least 0.01 wt % of an agent for treating a disease associated with oxidative modification of a protein in the composition. When intended for oral administration, this amount may be varied to be between 0.1 and about 70% of the weight of the composition. Preferred oral compositions contain between about 4% and about 50% of the agent for treating a disease associated with oxidative modification of a protein. Preferred compositions and preparations are prepared so that a parenteral dosage unit contains between 0.01 to 1% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the agent(s) for treating a disease associated with oxidative modification of a protein of from about 0.1 to about 10% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol.

In the methods of the invention, the agent(s) for treating a disease associated with oxidative modification of a protein may be administered through use of insert(s), bead(s), timed-release formulation(s), patch(es) or fast-release formulation(s).

It will be evident to those of ordinary skill in the art that the optimal dosage of the agent(s) for treating a disease associated with oxidative modification of a protein may depend on the weight and physical condition of the patient; on the severity and longevity of the physical condition being treated; on the particular form of the active ingredient, the manner of administration and the composition employed. It is to be understood that use of an agent for treating a disease associated with oxidative modification of a protein in a chemotherapy can involve such a compound being bound to an agent, for example, a monoclonal or polyclonal antibody, a protein or a liposome, which assist the delivery of said compound.

### EXAMPLES

The following Examples are offered by way of illustration and not by way of limitation.

### Example 1

### Preparation of Nitrotyrosine Peptides and Mass Spectrometry

Materials

Nitrotyrosine and trypsin were obtained from Sigma (St. Louis, Mo.). Tetranitromethane, ammonium bicarbonate, and sodium dithionite (or sodium hydrosulfite, Na₂S₂O₄) were obtained from Aldrich (Milwaukee, Wis.). The peptide, AAFGY(m-NO₂)AR, was obtained form Genosys (The Woodlands, Tex.) via custom commercial synthesis. HPLC-grade acetonitrile and water were purchased from Fisher Scientific (Pittsburgh, Pa.). Trifluoroacetic acid was obtained from Pierce (Rockford, Ill.). Bovine serum albumin (BSA) used to prepare nitrated BSA was obtained from Roche Molecular Biochemicals (Boehringer Mannheim, Indianapolis, Ind.). The MALDI matrices alpha-cyano-4-hydroxycinnamic and 2,5-hydroxy benzoic acid were obtained from Hewlett Packard (Palo Alto, Calif.) and Aldrich, respectively. Polyclonal and monoclonal anti-nitrotyrosine antibodies were purchased from Upstate Biotechnology (Lake Placid, N.Y.).

Methods

Nitration of BSA. Bovine serum albumin (7 mg/ml) was dissolved in 10 mM ammonium bicarbonate buffer and reacted with tetranitromethane in alcohol (Sokolovsky et al., 1967 Biochem. Biophys. Res. Commun. 27, 20-25). After reacting for one hour the reaction was quenched with acetic acid and the nitrated BSA was separated from nitroformate ion using a standard desalting column. The fraction obtained with similar retention time to the native BSA run for reference was lyophilized overnight. (Nitrated BSA was yellowish in appearance compared to unmodified BSA, which had no color). For the analysis of intact BSA samples (untreated and nitrated), protein was mixed with saturated sinapinic acid in 1:1 water/acetonitrile (v/v) and externally calibrated.

Tryptic hydrolysis and HPLC separation of N-BSA. Trypsin digestion of BSA and nitrated BSA proceeded at 37° C. with a trypsin/protein ratio of ˜1:20 (wt/wt) for 16 hours. The enzymatic digestion was quenched with phenylmethylsulfonyl fluoride (PMSF, Sigma) or frozen and lyophilized. The tryptic hydrolysate comprising both unmodified and modified peptides was then separated by reverse-phase HPLC. For off-line analysis, a Rainin (Woburn, Mass.) HPLC instrument was used. Peptides were eluted at a flow rate of 1 ml/min under gradient conditions; 10%-90% B in 60 min where solvent A consisted of 0.1% TFA in water and solvent B 0.08% TFA in 70% acetonitrile. Eluate was monitored at 210 nm (0.2 AUFS) with an ABI 785A absorbance detector (Perkin-Elmer, Inc., Applied Biosystems Division, Foster City Calif.) and fractions were collected.

Reduction of Nitrotyrosine to Aminotyrosine in situ. The reduction of nitrotyrosine to aminotyrosine was accomplished by adding as reducing agent sodium dithionite (Na₂S₂O₄) to peptide mixtures according to previously published methods (Sokolovsky et al., 1967). No cleanup was necessary to follow the complete conversion of all nitro groups to amines by MALDI-TOF, although a solid phase extraction cleanup using reverse-phase Zip-Tips® (Millipore) was sometimes employed to improve peptide signals under mass spectrometry analysis.

Immunoprecipitation and Immunoselection of Nitrotyrosine Proteins and Peptides. Nitrated BSA was immunoprecipitated with agarose-bound commercial anti-nitrotyrosine according to previous published procedures (MacMillan et al., 1999 Methods Enzymol 301, 135-45). Alternatively, peptides containing nitrotyrosine were selectively bound using this same agarose antibody or after conjugating free antinitrotyrosine to magnetic Dynabeads (Dynal, Lake Success, N.Y.) according to the manufacturer's protocols. Both tosyl-activated beads or hydrophobic uncoated magnetic beads (2.8-4.5 μM size) were used for this latter purpose. In this case the peptides were released by treating the beads with free nitrotyrosine, heat and/or dilute acetic acid.

Mass Spectrometry. All linear, reflectron and PSD spectra was taken on a PerSeptive Biosystems (Framingham, Mass.) DE-STR MALDI-TOF equipped with delayed extraction optics and a nitrogen laser. A prototype MALDI orthogonal quadrupole-TOF (or Q-TOF) based on a Mariner orthogonal TOF analyzer was used to obtain a collisional induced dissociation (CID) spectrum of the synthetic nitrotyrosine-containing peptide. This prototype Q-TOF instrument was also equipped with a standard nitrogen laser (337 nm) and data was acquired in the positive-ion mode with external calibration. In all cases, a 1 μL aliquot of each HPLC fraction was mixed with 33 mM alpha-cyano-4-hydroxycinnamic acid in acetonitrile/methanol (1/1; v/v) and air-dried on a gold-plated or stainless steel MALDI target. Mass spectra were acquired in the positive ion mode.

For ESI-MS analysis, peptides were analyzed on a Mariner orthogonal TOF mass spectrometer (PE Biosystems, Framingham, Mass.) equipped with an electrospray source. Peptide mixtures were analyzed as their nitrotyrosine derivatives or after conversion to their corresponding aminotyrosine analogs with reducing agent, or as mixtures of both. Typical solvents were water/methanol or water/acetonitrile for infused sample without upfront chromatography. For on-line HPLC ESI-TOF, the tryptic digest was separated on an ABI 140B solvent delivery system (Perkin-Elmer, Inc., Applied Biosystems Division, Foster City, Calif.) equipped with a Vydac (Hesperia, Calif.) C₁₈ (1×150 mm) column running at 50 μl/min under gradient conditions from 10%-60% B in 70 min, where solvent A consisted of 0.1% formic acid in H₂O and solvent B was 0.05% formic acid in ethanol/propanol (5/2; v/v). Mass spectra were acquired after 10:1 flow-splitting on a Mariner ESI-TOF mass spectrometer Mass accuracies of <100-50 ppm were obtained with external calibration and <10-50 ppm for MALDI-TOF with internal calibration.

Results

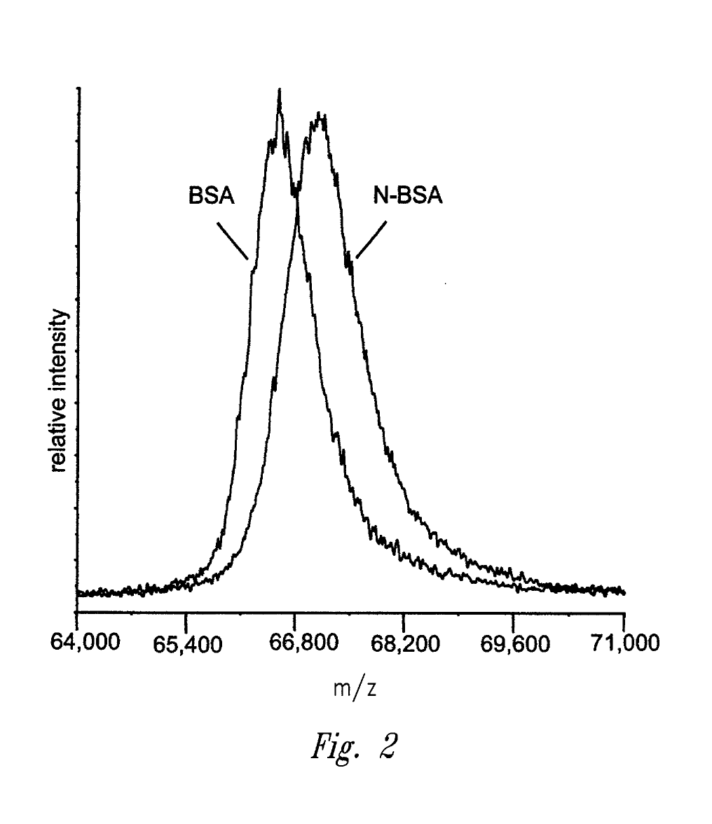

In this example, MALDI and electrospray ionization (ESI) spectrometry were used for the detection and characterization of nitrotyrosine modification at the peptide and protein level. When BSA was treated with tetranitromethane, the linear MALDI-MS spectrum of nitrated BSA showed a slight increase in the mass (˜420 Da) relative to unreacted BSA with a noticeable broadening and tailing of the unresolved isotope cluster (FIG. 2). According to non-limiting theory, the increase in mass observed for nitrated BSA appeared to result from nitration of some but not all tyrosine residues in BSA. Under MALDI conditions, mass spectra of several peptides generated from tryptic hydrolysis of bovine serum albumin (BSA) treated with tetranitromethane and the synthetic peptide AAFGY(m-NO₂)AR have shown that unique series of peaks were generated for peptides that contain the nitrotyrosine modification. In addition to the expected protonated molecular ions, (MH⁺), for select tyrosine-containing peptides that contain a nitro group modification, two other peaks at roughly equal abundance were observed at masses that would nominally correspond to the loss of one and two oxygen atoms, i.e., (M+H−16)⁺ and (M+H−32)⁺. These secondary peaks were not formed by direct fragmentation, but appeared to be photochemical reaction products formed by exposure to the N₂ laser (337 nm) prior to acceleration. In addition to these major peaks, two much less abundant, minor peaks were also seen at positions that were 14 and 30 Da less than the expected intact nitrotyrosine molecular ion, consistent with the photoreduction of nitrotyrosine to hydroxylaminetyrosine and aminotyrosine, respectively (Scheme 2).

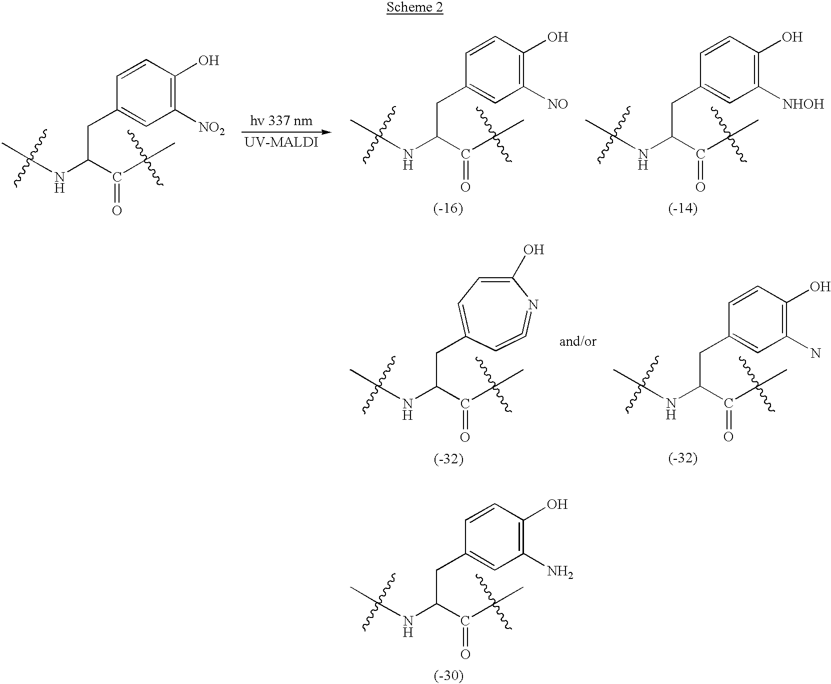

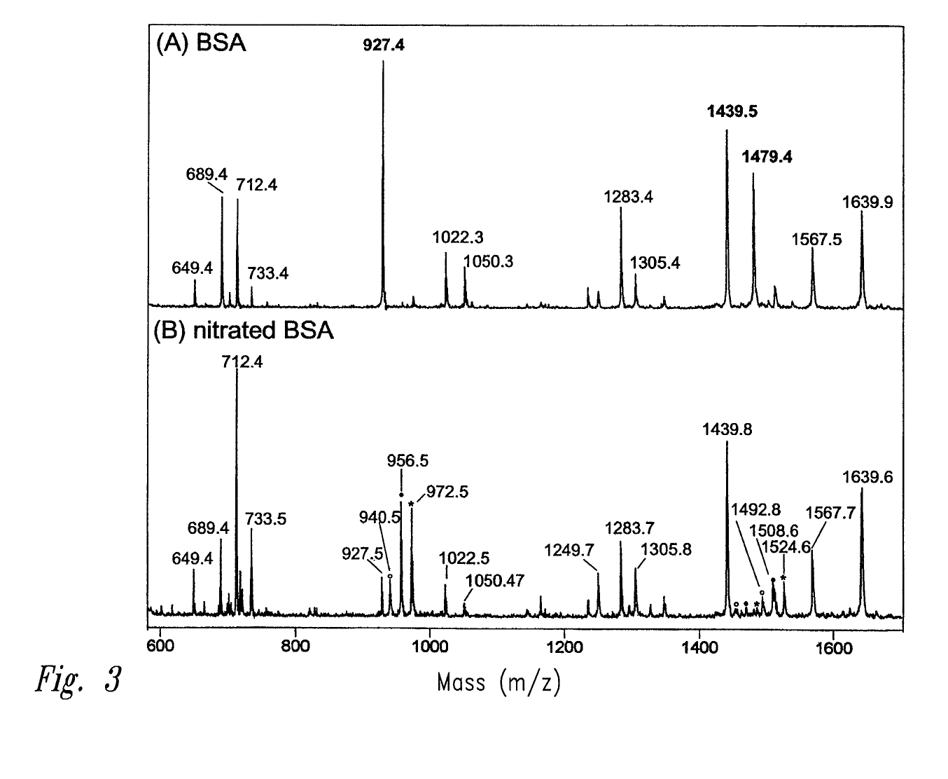

Following tryptic hydrolysis of nitrated BSA, a clear difference between the peptide maps of the unseparated hydrolyzate with and without nitration was apparent after analysis by MALDI-TOF (FIG. 3). In these spectra, two peaks matching positions of predicted tryptic peptides of (untreated) BSA that contained tyrosine, at m/z 927.4 (MH⁺=YLYEIAR) and 1479.4 (MH⁺=LGEYGEQNALIVR) were largely absent in the MALDI-MS tryptic map of the nitrated BSA sample. Instead, peaks were seen that correspond to the substitution of a hydrogen with NO₂ (ΔM=45 Da) at m/z 972.4 and 1524.6. respectively. Two additional peaks associated with the expected molecular ion peak were also observed that were 16 and 32 Da lower in mass. As shown in the inset in FIG. 3, these peaks were at m/z 956.5 and 940.5 for the 972.4 peaks, and m/z 1508.6 and 1492.8 for the 1524.6 peaks.

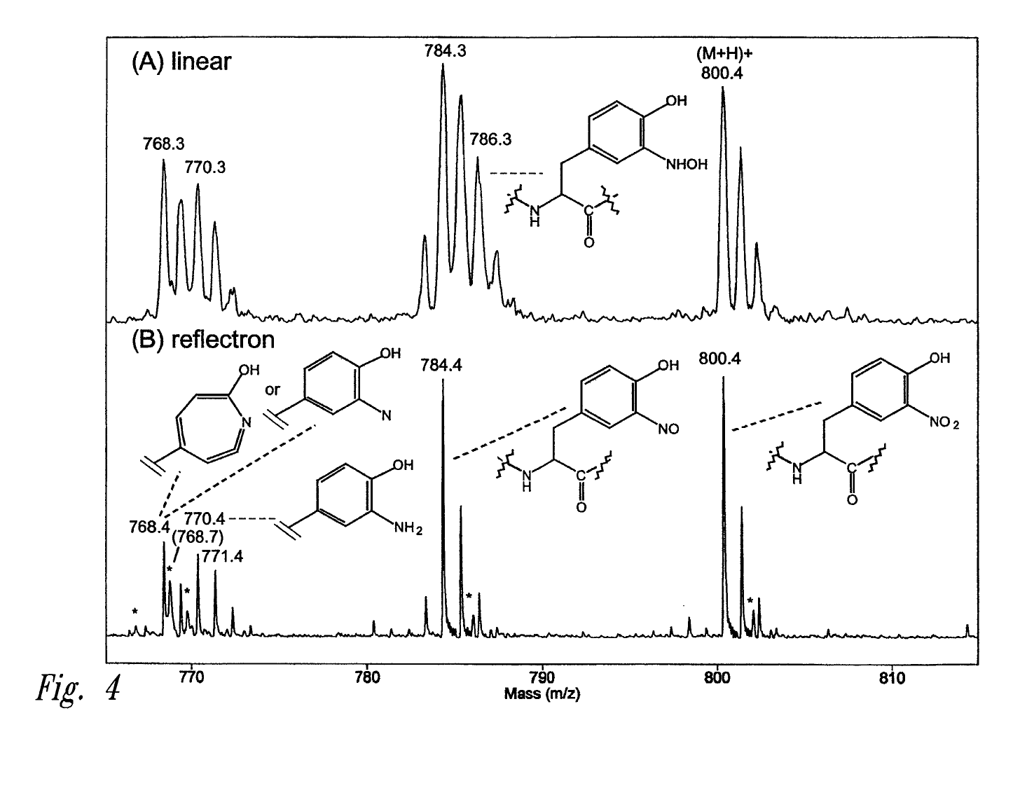

Analysis of a synthetic nitrotyrosine-containing peptide, AAFGY(m-NO₂)AR, also revealed a similar set of molecular and pseudomolecular ions (FIG. 4). Quantitative conversion of this synthetic peptide containing nitrotyrosine to the amino derivative (i.e., 3-aminotryosine) in situ with Na₂S₂O₄ yielded a single molecular ion peak with high relative abundance. Likewise, reduction of the tryptic hydrolyzate of nitrated BSA with sodium dithionite also showed a shift to a single peak 30 Da lower in mass than the corresponding nitrated peptides, i.e., m/z 972.4-->942.4 and m/z 1524.6-->1494.8. In addition, the other peaks associated with the nitrotyrosine-containing peptides at masses 16 and 32 Da lower in mass were absent.

Similarly, under electrospray ionization conditions, the reduced aminotyrosine peptide had a detectable mass that was 30 Da lower than the corresponding nitrotyrosine-containing peptides. Therefore, in all cases, the aminotyrosine derivative yielded a single peak that corresponded to the expected mass for this peptide. The 30 Da mass difference between the nitrotyrosine and aminotyrosine-containing peptides provided another unique mass signature pattern for nitrotyrosine-containing peptides and was a general attribute of all peptides containing this modification. Indeed, when a 1/1 mixture of the synthetic nitrotyrosine and reduced aminotyrosine peptide was analyzed by electrospray ionization (ESI) mass spectrometry, both peaks were observed. Under ESI-HPLC/MS conditions, the aminotyrosine-containing peak eluted prior to the nitrotyrosine-containing analog since it was more hydrophilic.

(The mass differential between aminotyrosine and nitrotyrosine may be exploited according to an experimental protocol wherein half the peptide mixture is reduced with Na₂S₂O₄, then added back and mixed with the untreated peptide sample, and analyzed by ESI-HPLC/MS and MS/MS. A peak would be identified as containing nitrotyrosine only if an analogous earlier eluting peak 30 Da lower in mass was also observed. One could then select these peaks for MS/MS analysis in a second experiment, or construct a simple data-dependent algorithm to make this decision during the initial HPLC experiment itself.) Analysis of several of the peptides containing nitrotyrosine, including the synthetic peptides AAFGY(m-NO₂)AR, by both (i) post source decay (PSD) on a conventional MALDI-TOF instrument, and (ii) a MALDI collisionally activated spectra taken on a quadrupole-orthogonal TOF instrument, yielded extensive fragmentation that allowed identification of both the position and modification of the nitrated tyrosine residue.

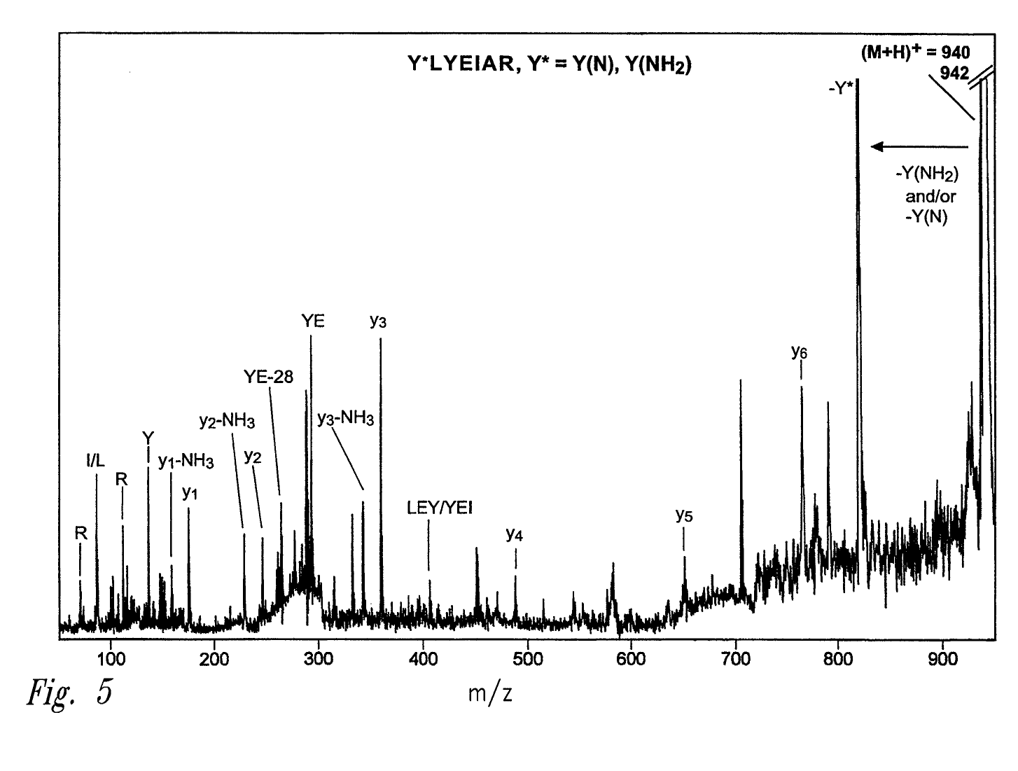

Purified peptide fractions of the tryptic hydrolysate of nitromethane-treated BSA was subsequently obtained by HPLC separation with monitoring at 450 nm to identify ‘nitrated’ tryptic peptides. Several peptides were subsequently isolated and found by mass, and by the mass signature pattern, to contain nitrotyrosine. MALDI PSD analysis of one of these peptides showed that the peptide at m/z 972.4 was YLYEIAR, where one of the two tyrosine residues contained the nitro group modification. The PSD spectrum, where the timed ion selection passed the photodecomposition product peaks at m/z 942 and 940, clearly showed that the first tyrosine is nitrotyrosine, Y(NO₂)LYEIAR (see FIG. 5). In the resulting MALDI-PSD spectrum, the presence of a complete y-ion series established that the first tyrosine residue (tyrosine-161) contained the nitrogroup modification, Y¹⁶¹(NO₂)LYEIAR¹⁶⁷. Consistent with this determination was the absence of doublet ions separated by 2 Da in the y-ion series, and doublets corresponding to the y₅ and y₆ appeared if the second internal tyrosine (tyrosine-163) was nitrated. Of eight tyrosine residues representing potential nitration sites that were analyzed by the MALDI-MS peptide spectra, only two were shown to be significantly nitrated, indicating that there is selectivity of tyrosine nitration by tetranitromethane. Similar findings have been reported from studies of in vitro nitration of SOD, glutamine synthetase and other proteins as described above.

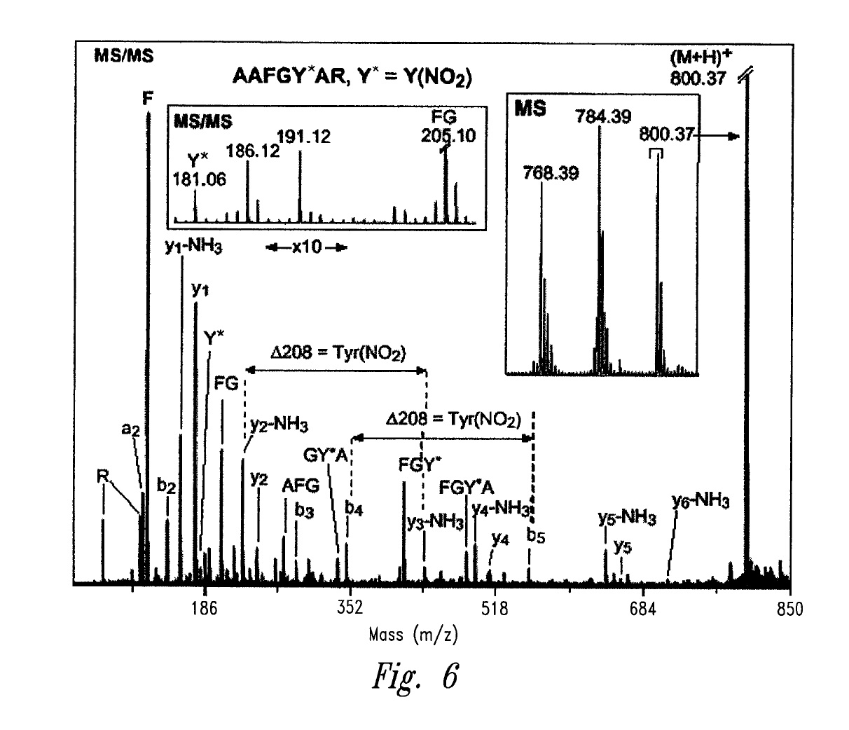

Similarly, the synthetic nitrotyrosine-containing peptide AAFGY(m-NO₂)AR was subjected to CID analysis using a quadrupole-orthogonal TOF mass spectrometer with a MALDI source (FIG. 6). Three dominant ions were observed in the spectrum at m/z 800.4, 784.4 and 768.4 (the expected molecular ion triplet). In this case, the ion at 800.4 of the (M+H)⁺ peaks was selected by the quadrupole analyzer, collisionally activated, and the resulting fragments separated on the TOF analyzer. In the resulting MS/MS spectrum, no peaks were seen corresponding to the loss of 16 or 32, as expected if these were photo-decomposition fragments and not thermal fragments. A single immonium ion peak was seen at m/z 181.1 corresponding to nitrotyrosine, ⁺H₂N═CHCH₂C₆H₃(OH)(NO₂). In addition a series of fragment ion peaks corresponding to the y- and b-ion series, allowed for the complete sequence determination of this peptide. For example, the b₄ and b₅ ions (cleavage at the amide linkage with charge retention at the N-terminus) were clearly visible at m/z 347.2 and 555.2, respectively, yielding the unique mass difference of a nitrotyrosine residue (208 Da).

Both the PSD data obtained on the DE-STR and collisional activation spectrum using the quadrupole-orthogonal-TOF showed that the breakdown of the peptide occurred during the ionization process and not while the ion was in the flight tube. If these additional peptide molecular ion peaks were fragment generated after ionization and acceleration (i.e., post-source decay fragments), the selected ion window for the (M+H)⁺ of the nitrated peptide would show additional peaks at (M+H−16)⁺ and (M+H−32)⁺. This was not the case, suggesting that these were fragments generated from the UV-laser prior to ion extraction and acceleration.

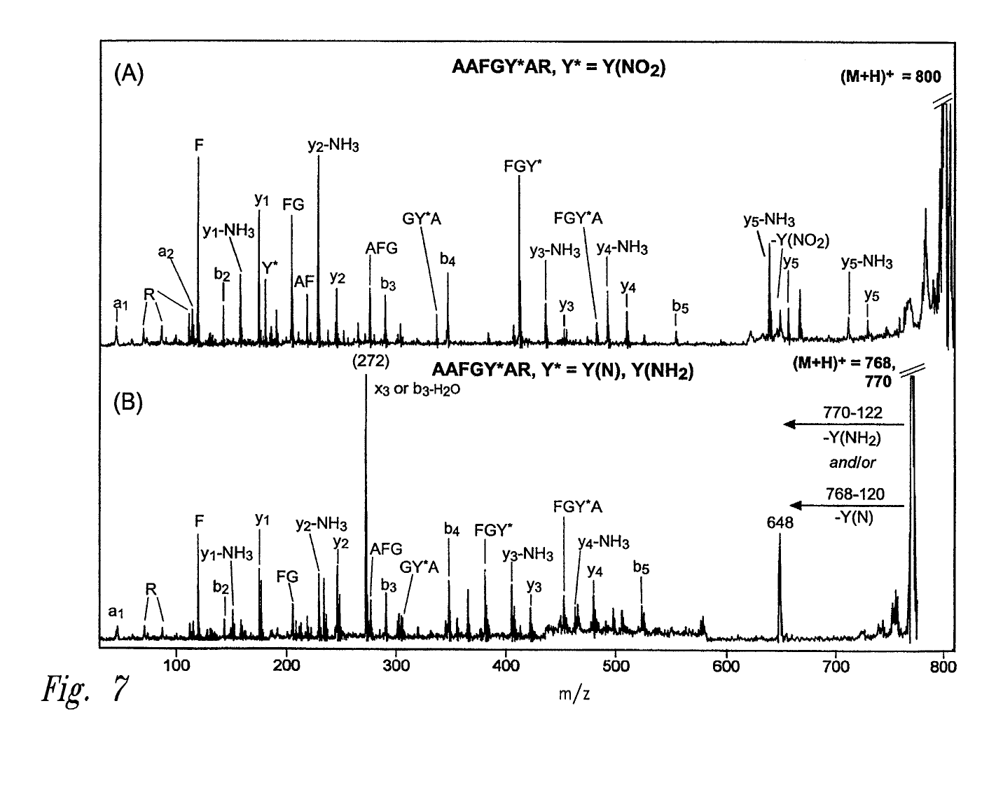

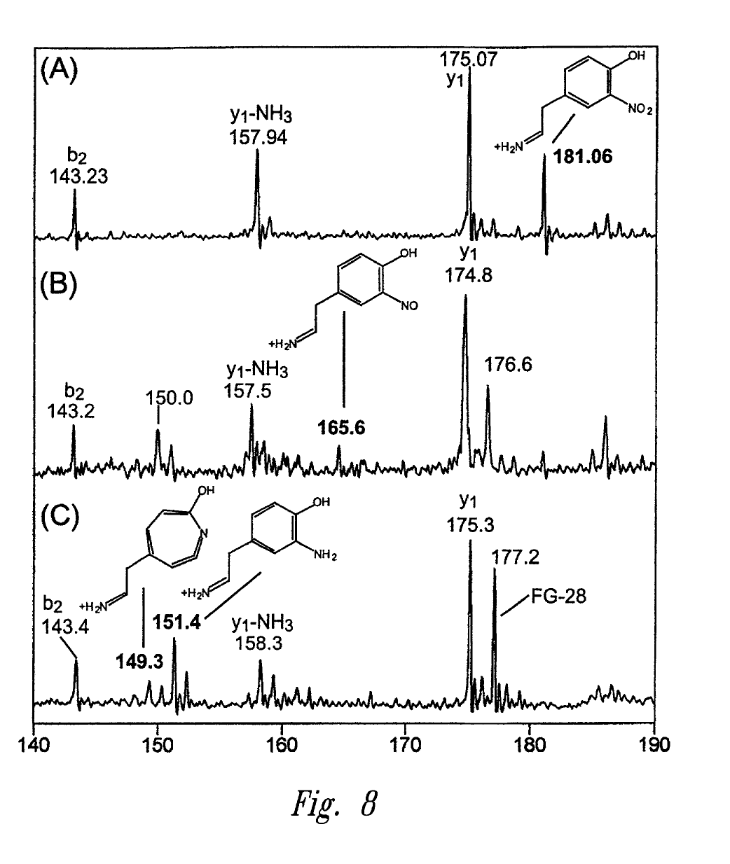

PSD data were taken on each of the three molecular ion triplet peaks of the nitrated synthetic peptide analyzed in FIG. 6, with some limitations due to the fact the timed ion selection window was not narrow enough in all cases to pass each one exclusively (see FIG. 7). For example, timed-ion mass selection windows could be established for MH⁺ and (M+H−32)⁺ peaks, not for the (M+H−16)⁺. Immonium ion peaks originating from photochemical product of the parent (M+H−32)⁺ peak of the nitrotyrosine-containing peptide were evident at m/z 149 and 151 (FIG. 8). An immonium ion peak from nitrotyrosine from the (M+H)⁺ peak of nitrotyrosine was also evident at m/z 181. Immonium ions were evident for all three peaks for the (M+H−16)⁺ peak due to the partial timed-ion passage of the neighboring peaks (i.e., (M+H)⁺ and (M+H−32)⁺), but also yielded one new immonium ion peak at m/z 165 that presumably originated from the (M+H−16)⁺ peak only (Scheme 3).

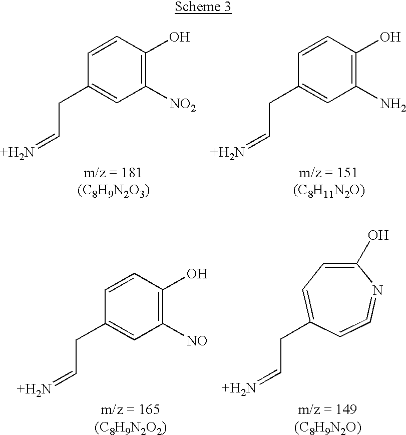

Therefore, under standard MALDI conditions, using a nitrogen laser, a specific photochemical reaction has been demonstrated for peptides containing nitrotyrosine. Under positive ionization conditions, in addition to the expected molecular ions (M+H)⁺, two prominent additional mass peaks were observed that were 16 and 32 Da lower in mass, and that corresponded to the loss of one and two oxygen species, respectively. In addition, fragment immonium ions at low mass were observed that recapitulated this process, giving rise to the expected immonium ion for nitrotyrosine at m/z 181 (+H₂N═CHCH₂C₆H₃(OH)m(NO₂)) as well as immonium ions for photo-decomposition products at m/z 165 (+H₂N=CHCH₂C₆H₃(OH)m(NO)), m/z 151 (+H₂N═CHCH₂C₆H₃NH₂(OH), and at m/z 149 (+H₂N═CHCH₂C₆H₃N(OH). This unique molecular ion signature provided unequivocal evidence regarding the presence or absence of nitrotyrosine in a given peptide. The invention thus contemplates identification of these modifications in the presence of complex peptide mixtures, and further at the protein (as distinguished from peptide) level, provided the mass spectrometer used has sufficient resolution to separate out the isoforms (e.g., tyrosine nitrated variants) of this protein with these mass differences (i.e., Δ16 Da at the mass of the protein, which may be anywhere from 10,000-100,000 Da or larger).

As shown above, the unique immonium ion fragments in the low mass region of PSD and other types of MS/MS or collisionally activated spectrum also provided evidence for the presence of nitrotyrosine in a peptide or an intact protein.

### Example 2

### Time, Laser Flux and Concentration Dependence of MALDI Photodecomposition of Nitrotyrosine-Containing Peptides

Analysis of the concentration dependence of the synthetic peptide AAFGY(m-NO₂)AR were conducted using a 7.5 mM solution of the peptide prepared in water (0.6 mg/O/1 mL), with aliquots diluted in successive 10-fold increments (serial dilution) down to 7.5 nM. In each case, 1 microliter of the peptide solution was mixed with 2 microliters of matrix (33 mM α-cyano-4-hydroxycinnamic acid), and 1 microliter was deposited on the MALDI target covering a sample range of 2.5 nmol to 2.5 fmol of total peptide spotted.

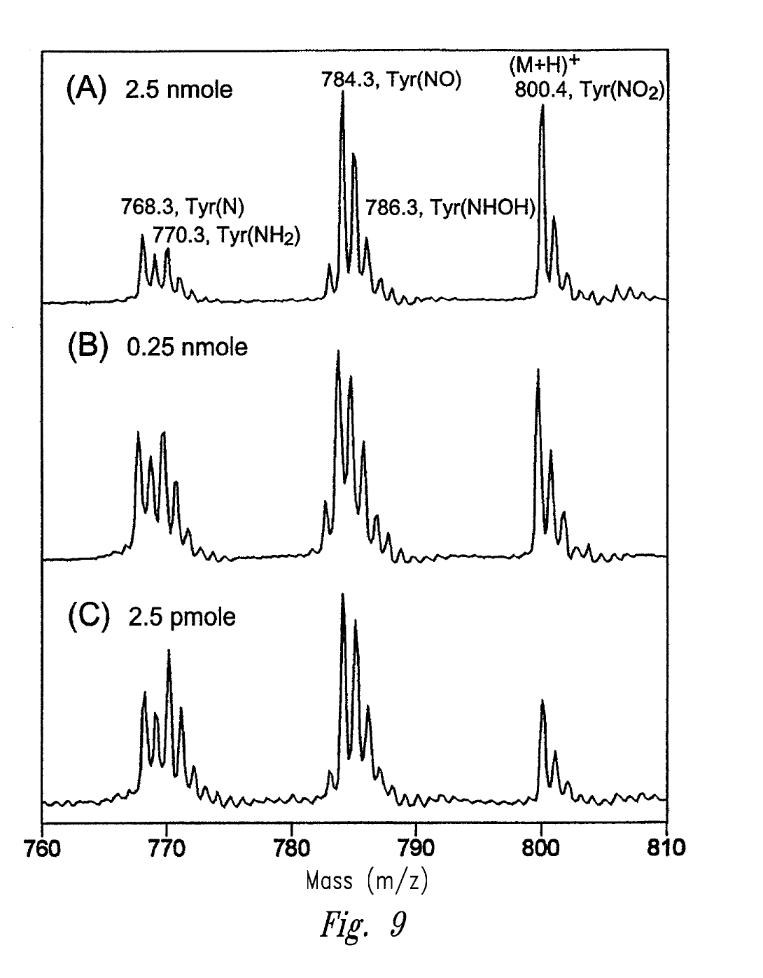

To examine the effects of time (i.e., number of laser shots), laser power, and peptide concentration on the formation of photodecomposition fragments, a series of linear MALDI-MS experiments was carried out using the synthetic peptide (AAFGY(m-NO₂)AR) and the effects of timing or numbers of laser shots on the relative abundance of the molecular ion and photodecomposition products were examined by integrated successive sets of laser pulses at the same spot (16 shots each). No difference was observed between the first spectrum (first 16 shots) and successive sets of integrated spectra (e.g., shots 17-32, 33-48, etc.). Similarly, laser power appeared to have no discernable effect on the relative abundances of these species—an increase in laser fluence from threshold levels, and higher levels, increased both the molecular ion and the products to the same extent. A marked difference in the molecular ion region was observed, however, when the peptide concentration (or amount of peptide spotted) was varied (FIG. 9). At very high amounts (2.5 nmol/1L, FIG. 9A) the −30/32 Da photodecomposition products [Tyr(NH₂) and Tyr(N)] were considerably lower in abundance than both the molecular ion and the −14/16 Da products [Tyr(NHOH) and Tyr(NO)]. At successive 10-fold dilutions to 2.5 pmol, the abundance of the −30/32 Da photodecomposition product ions steadily increased to levels approximately equal to the −14/16 products, whereas the relative abundance of the molecular ion decreased 2-3 fold. At even lower sample loadings (e.g., 0.25 pmol to 25 fmol), the relative abundances of the parent and product ions remained essentially identical to those obtained at the 2.5 pmol peptide level (see FIG. 9C).

### Example 3

### In Situ Reduction of Nitrotyrosine Containing Peptides

Materials and methods as described in Example 1 are used to identify tyrosine nitrated peptides and confirm their identities by reductive conversion to aminotyrosine peptides. In situ reduction of nitrotyrosine-containing peptides with Na₂S₂O₄ quantitatively converts these peptides to their amino-tyrosine analogs, which are characterized by MS. These aminotyrosine peptides are 30 Da lower in mass than the corresponding nitrotyrosine peptides and do not undergo photo-decomposition reactions under MALDI conditions. Under ESI-MS conditions, both the nitrotyrosine and amino-tyrosine peptides give rise to single masses, but differ by 30 Da as expected from the difference in their molecular weights. Therefore, by examining molecular ion profiles of peptides before and after reduction, one can examine in a data-dependent fashion, in real time switching between MS and MS/MS modes or in an off-line mode, the tyrosine-containing peptides that are modified. Several scenarios are contemplated:

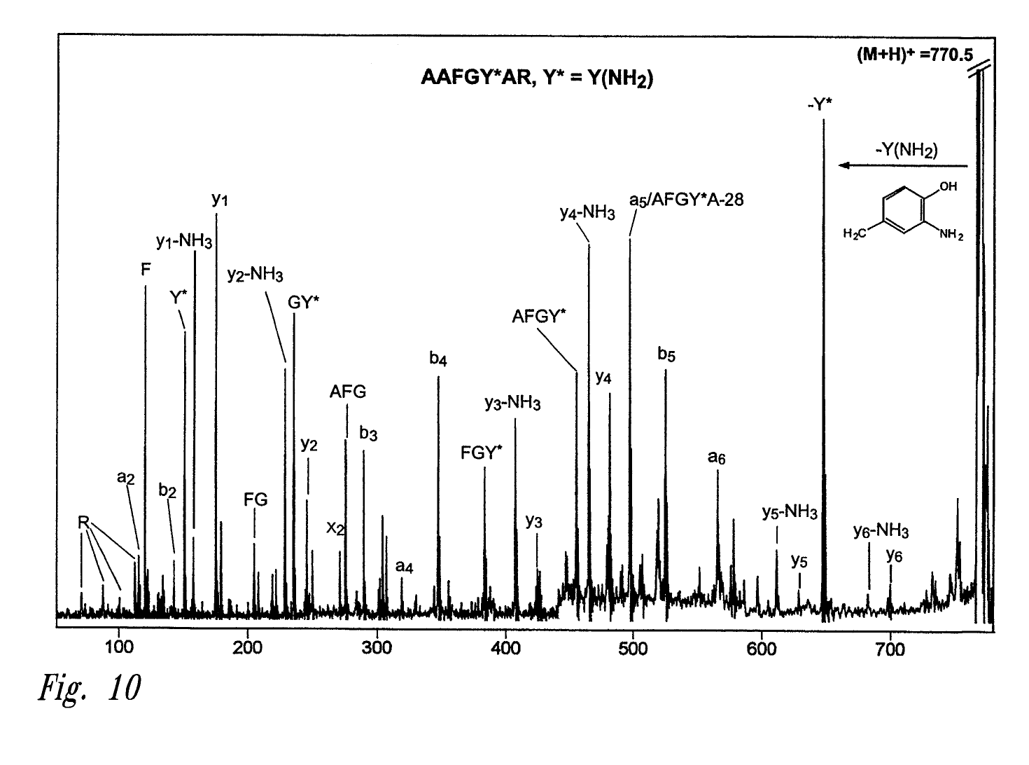

(1) Unseparated or HPLC fractionated digests of proteins characterized by MALDI-MS are compared for the identification of nitrotyrosine-containing peptides. The triplet series of molecular ions (i.e., MH⁺, (M+H−16)⁺ and (M+H−32)⁺) is detected for a nitrotyrosine-containing peptide and shifted down in mass by 30 Da for the corresponding reduced aminotyrosine-containing analog. These peptides are then selected for PSD or other type of fragmentation sequence to gain additional amino acid sequence information. For example, in FIG. 10 a PSD spectrum is shown for the reduced version of the peptide AAFGY(NH₂)AR. PSD analysis of the molecular ion for the synthetic peptide (FIG. 10) resulted in a spectrum that recapitulated many features seen in the MALDI-PSD spectrum of the photodecomposition products of the nonreduced nitrotyrosine peptide at m/z 768 and 770 (cf FIGS. 5 and 7B). In addition to abundant y- and b-type ion series, the most abundant fragment was the sidechain loss of aminotyrosine, which was also the major fragment observed for the photodecomposition product spectrum.

Sequence information so obtained is queried using any of several commercially available proteomics search routines, such as that made available by the University of California, San Francisco (http://prospector.ucsf.edu/, MS-Seq mode) to identify the specific peptide sequence. Such information is useful to identify the source of the specific peptides (protein identity) and the specific tyrosine residue position of modification.

(2) Online HPLC-MS analysis under ESI conditions is used instead of off-line MALDI methods, and peptide mixtures containing one of (a) the nitrotyrosine peptides, (b) the reduced aminotyrosine peptides analogs, or (c) a mixture of (a) and (b) are analyzed directly. Only peptides that undergo the 30 Da mass shift are identified as containing nitrotyrosine, and this information can be used to select molecular ions for tandem MS/MS sequence analysis. Such data-dependent protocols exist for several mass spectrometer platforms including ion-traps (e.g., LCQ from Finnigan), quadrupole-orthogonal-TOF mass spectrometers (e.g., PE-Sciex Q-STAR or Micromass Q-Tof), or triple quadrupoles (available from many vendors).

(3) For both of the above protocols, a protein fraction enriched in protein containing the nitrotyrosine modification is obtained by employing standard immunoprecipitation methods (e.g., MacMillan et al., 1999). Proteins are immunoprecipitated using agarose-bound polyclonal or monoclonal antibodies. The immunoprecipitate is directly applied to SDS-PAGE gels following solubilization in an appropriate buffer, and components are separated according to size. From the SDS-PAGE gel specific bands are then excised, subjected to in situ trypsin digestion (or digestion with another protease), extracted and analyzed by mass spectrometry using techniques for proteomics analysis as described above (e.g., Shevchenko et al., 1996; Matsui et al., 1997; Wong et al., 1999). Peptides containing nitrotyrosine are then identified as described above, using MALDI or ESI-MS instruments.

### Example 3

### Cybrid Studies

To construct cytoplasmic hybrid or “cybrid” cell lines containing mtDNA from the human volunteers, SH-SY5Y neuroblastoma cells were depleted of mitochondrial DNA, and fused with patient platelets as described by Miller et al. (1996 J. Neurochem 67:1897-1907; see also U.S. Pat. No. 5,888,498). Briefly, from 6 ml of citrate-anticoagulated blood drawn from human subjects as described above, platelets were isolated by differential centrifugation. The cell pellet was resuspended in 1 ml calcium-free Minimal Essential Medium (MEM; Gibco BRL, Grand Island, N.Y.). ρ⁰ SH-SY5Y cells were harvested from a 75 cm² flask by trypsinization, resuspended in 10 ml calcium-free MEM, and collected by centrifugation at 200 g for 5 minutes. The ρ⁰ cell pellet was resuspended in 1 ml calcium-free MEM. The platelet suspension was added to the ρ⁰ cell suspension, mixed gently, and the mixture was incubated 5 min at room temperature. The cells were collected by centrifugation at 400 g for 5 min. To promote fusion, 150 μl polyethylene glycol-1000 solution (50% w/v in calcium-free MEM; J. T. Baker, Phillipsburg, Pa.) was added with gentle mixing using a pipet. The mixture was incubated 1.5 min at room temperature, then diluted with 12 ml p° culture medium (Dulbecco's Modified Eagle Medium [Irvine Scientific, Irvine, Calif.], 10% fetal calf serum [Irvine Scientific, Irvine, Calif.], 1 mM sodium pyruvate, 50 μg/ml uridine, and 100 U/ml penicillin/streptomycin solution (Gibco BRL, Grand Island, N.Y.). The fused cells were transferred to a tissue culture flask and grown in a humidified 5% CO₂, 95% air environment at 37° C. The medium was changed daily. After 1 week, selection medium (ρ⁰ medium lacking uridine and pyruvate) was substituted for the ρ⁰ medium. The cybrid cells were allowed to grow and repopulate their mitochondrial DNA for 6-8 weeks before use. Cybrid cells were harvested by scraping in phosphate buffered saline (PBS, Irvine Scientific, Irvine, Calif.). Submitochondrial particles (SMP) were prepared from the cells as described below for individual enzyme assays.

Enzyme activities of citrate synthase and of mitochondrial electron transport chain complexes I and IV were measured as described by Miller et al. (1996) and Parker et al. (1994 Neurology 44:1090-1096). Brief descriptions of the assays follow:

To determine citrate synthase activity in cultured cybrid cells produced as described above, 2×10⁵ cells were added to a spectrophotometer cuvette for each group. For citrate synthase determination in clarified skeletal muscle homogenate prepared as described above, 20 μg of “total lysate” was added to each cuvette. Assay buffer (0.04% Triton X-100, 0.1 mM 5,5′-dithio-bis(2-nitrobenzoic acid), 100 mM Tris, pH 8.0) pre-warmed to 30° C. was added to each cuvette. Acetyl CoA (final concentration 50 μM) and oxaloacetic acid (final concentration 500 μM) were added to bring the assay volume to 1 ml. The change in absorbance at 412 nm was measured for 3 min. in a Beckman DU7400 spectrophotometer (Beckman Instruments, Palo Alto, Calif.).

Complex I (NADH:ubiquinone oxidoreductase) in cultured cells: Cell suspensions (2 million cells/ml) were incubated with 0.005% digitonin in HBSS containing 5 mM EDTA (HBSS/EDTA) for 20 seconds at room temperature. Fifty volumes HBSS/EDTA were then added. The solution was centrifuged at 14,000 g for 10 min. at 4° C., and the pellet resuspended in HBSS/EDTA containing 1 μM pepstatin, 1 M leupeptin and 100 μM phenylmethylsulfonyl fluoride (PMSF). The resultant solution was sonicated for 6 minutes on ice in a cup-horn sonicator (Sonifier 450: Branson, Danbury, Conn.) at 50% duty cycle, 50% power. An aliquot of the solution (30-100 μg protein) was added to a 1 ml cuvette. Coenzyme Q1 (0.042 mM final concentration), NADH (0.1 mM final concentration), and assay buffer (25 mM potassium phosphate, 0.25 mM EDTA, 1.5 mM potassium cyanide, pH 8.0) were added. The change in absorbance at 340 nM was measured for 2 minutes. Rotenone (2.5 μM final concentration) was added, and a second 2 minute reading was taken. Activity was calculated as the rate in the absence of rotenone minus the rate in the presence of rotenone.

Complex IV (Cytochrome c oxidase) in cultured cells: The SMP solution was prepared as described for Complex 1. Assay buffer (20 mM potassium phosphate, pH 7.0), SMP (1-50 μg protein), n-dodecyl-β-D-maltoside (0.1 mg/ml final), and cytochrome c (5 mM) were added to a cuvette in a total volume of 1 ml. The change in absorbance of reduced cytochrome c at 550 nm was measured for 90 seconds. The cyanide-inhibited rate was subtracted to yield activity.

Complex IV in skeletal muscle: SMP were prepared as described above. This preparation was then substituted for the cultured cell preparation in the Complex IV assay described above.

Complex V (ATP synthase) activity was measured using a coupled spectrophotometric assay as follows: SMP were incubated in assay buffer containing 1 mM ATP, 1 mM phosphoenolpyruvate, 0.3 mM NADH, 3 U/ml pyruvate kinase, and 10 U/ml lactate dehydrogenase at 30° C. The change in absorbance at 340 nm was measured for 5 min in a Beckman DU 7400 spectrophotometer. The ATP synthase activity was expressed as nmoles NADH oxidized per minute per mg lysate or SMP protein.

Reactive oxygen species production: Production of reactive oxygen species by cybrid cells in culture was measured using the fluorescent dye dichlorodihydrofluorescein (Molecular Probes, Eugene, Oreg.) as described by Miller et al. (1996). Cells were plated at 75,000 cells per well in 96-well plates and allowed to grow overnight in a 5% CO₂, 95% air, humidified 37° C. incubator. The cells were rinsed with HBSS, then incubated with HBSS containing 30 μM 2′,7′-dichlorodihydrofluorescein diacetate (Molecular Probes, Eugene, Oreg.) for 2 hr. After rinsing with HBSS, the fluorescence was measured using a Cytofluor model 2350 plate reader (Millipore, Bedford, Mass.) with excitation at 485 nm and emission at 530 nm.

Western Blots: Antibody sources were as follows: Antibodies specific for ETC Complex IV, subunits I, II and IV, were from Molecular Probes, Inc. (Eugene, Oreg.); antibodies specific for ATP synthase subunit 8 were generously provided by Dr. Russell Doolittle (Univ. California San Diego). Equal amounts of SMP protein or “total lysate” from skeletal muscle biopsy preparations or from cultured cells, prepared as described above, were subjected to SDS polyacrylamide gel electrophoresis on 4-10% gels (Novex, San Diego, Calif.). The proteins were electroblot transferred to Hybond ECL nitrocellulose (Amersham, Buckinghamshire, England) using standard procedures, and probed with each of the above antibodies. Bands were visualized using an ECL Western Blot Analysis System (Amersham, Buckinghamshire, England) according to the supplier's instructions. Band densities were measured by scanning the autoradiograms, and quantitative data obtained from the scans using National Institutes of Health Image Analysis software (NIH, Bethesda, Md.)

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for identifying oxidative modification of a protein, comprising:
generating a mass spectrum of all or a portion of a protein fraction derived from a biological sample, the protein fraction comprising at least one peptide that includes a nitrotyrosine residue, wherein determination of nitrotyrosine in said sample indicates the protein is oxidatively modified.

2. A method for identifying oxidative modification of a protein, comprising:
comparing (i) a first mass spectrum of all or a portion of a first protein fraction derived from a first biological sample, said first protein fraction comprising at least one peptide that includes a nitrotyrosine residue, to (ii) a second mass spectrum of all or a portion of a second protein fraction derived from a second biological sample, wherein determination of nitrotyrosine in said second protein fraction indicates that a protein therein is oxidatively modified.

3. A method for identifying oxidative modification of a protein, comprising:
contacting all or a portion of a protein fraction derived from a biological sample with at least one proteolytic agent under conditions and for a time sufficient to generate a plurality of peptide fragments derived from said protein fraction, the protein fraction comprising at least one peptide that includes a nitrotyrosine residue; and
generating a mass spectrum of one or more of said peptide fragments, wherein determination of nitrotyrosine in at least one of said peptide fragments indicates that a protein in the biological sample is oxidatively modified.

4. A method for determining protein tyrosine nitration in a subject, comprising:
isolating at least one protein comprising nitrotyrosine from a biological sample derived from a subject;
contacting the protein with at least one proteolytic agent under conditions and for a time sufficient to generate a plurality of peptide fragments derived from said protein; and
comparing a mass spectrum of one or more of said peptide fragments to a mass spectrum of a control sample containing nitrotyrosine, and therefrom determining protein nitration in the subject.

5. The method of any one of claims 1-4 wherein the mass spectrum is generated by matrix assisted laser desorption ionization mass spectrometry.

6. The method of claim 5 wherein determination of nitrotyrosine comprises detection in the mass spectrum of (a) a peptide comprising nitrotyrosine; (b) a peptide comprising nitrotyrosine that lacks one oxygen atom; and (c) a peptide comprising nitrotyrosine that lacks two oxygen atoms.

7. The method of any one of claims 1-4 wherein the mass spectrum is generated by matrix assisted laser desorption ionization time-of-flight mass spectrometry, and wherein determination of nitrotyrosine comprises detection in the mass spectrum of (a) a peptide comprising nitrotyrosine; (b) a peptide comprising nitrotyrosine that lacks one oxygen atom; and (c) a peptide comprising nitrotyrosine that lacks two oxygen atoms.

8. A method for identifying oxidative modification of a protein, comprising:
comparing (a) a first mass spectrum of a first portion of a protein fraction derived from a biological sample, wherein the protein fraction comprises at least one peptide that includes a nitrotyrosine residue, to (b) a second mass spectrum of a second portion of the protein fraction derived from the biological sample, wherein the second mass spectrum is generated subsequent to exposure of said second portion to conditions sufficient to convert nitrotyrosine to aminotyrosine, wherein the second portion of the protein fraction comprises at least one peptide that includes an aminotyrosine residue derived from nitrotyrosine, and wherein determination of nitrotyrosine in said first portion and of aminotyrosine in said second portion indicates that at least one protein in the biological sample is oxidatively modified.

9. The method of claim 8 wherein prior to the step of comparing, the protein fraction is contacted with at least one proteolytic agent under conditions and for a time sufficient to generate a plurality of peptide fragments derived from said protein fraction.

10. The method of claim 8 wherein the peptide that includes an aminotyrosine residue derived from nitrotyrosine undergoes sidechain loss of aminotyrosine.

11. A method for identifying oxidative modification of a protein, comprising:
comparing (a) a first mass spectrum of a first portion of a protein fraction derived from a biological sample, wherein the protein fraction comprises at least one peptide that includes a nitrotyrosine residue, to (b) a second mass spectrum of a second portion of the protein fraction derived from the biological sample, wherein the second mass spectrum is generated subsequent to contacting said second portion with sodium dithionite under conditions and for a time sufficient to convert nitrotyrosine to aminotyrosine, wherein the second portion of the protein fraction comprises at least one peptide that includes an aminotyrosine residue derived from nitrotyrosine, and wherein determination of nitrotyrosine in said first portion and of amino tyrosine in said second portion indicates that at least one protein in the biological sample is oxidatively modified.

12. The method of claim 11 wherein prior to the step of comparing, the protein fraction is contacted with at least one proteolytic agent under conditions and for a time sufficient to generate a plurality of peptide fragments derived from said protein fraction.

13. The method of claim 11 wherein the peptide that includes an aminotyrosine residue derived from nitrotyrosine undergoes sidechain loss of aminotyrosine.

14. A method for detecting in a subject the presence of, or risk for having a disease associated with oxidative modification of a protein, comprising:
generating a mass spectrum of all or a portion of a protein fraction of a biological sample derived from a subject suspected of having or being at risk for having a disease associated with oxidative modification of a protein, the protein fraction comprising at least one peptide that includes a nitrotyrosine residue, wherein determination of nitrotyrosine in said sample indicates the protein is oxidatively modified, and therefrom detecting risk for or presence of a disease in the subject.

15. A method for detecting in a subject the presence of, or risk for having a disease associated with oxidative modification of a protein, comprising:
comparing (i) a first mass spectrum of all or a portion of a first protein fraction of a biological sample derived from a first subject suspected of having or being at risk for having a disease associated with oxidative modification of a protein, said first protein fraction comprising at least one peptide that includes a nitrotyrosine residue, to (ii) a second mass spectrum of all or a portion of a second protein fraction of a biological sample derived from a second subject known to be free of a presence or risk for having a disease associated with oxidative modification of a protein, said second protein fraction lacking nitrotyrosine, wherein determination of the presence of nitrotyrosine in said first protein fraction and the absence of nitrotyrosine in said second protein fraction indicates risk for having or presence of a disease in the first subject.

16. A method for identifying a protein that is oxidatively modified in a disease associated with oxidative modification of a protein, comprising:
comparing (i) a first mass spectrum of all or a portion of a first protein fraction of a biological sample derived from a first subject having or being at risk for having a disease associated with oxidative modification of a protein, said first protein fraction comprising at least one peptide that includes a nitrotyrosine residue, to (ii) a second mass spectrum of all or a portion of a second protein fraction of a biological sample derived from a second subject known to be free of a presence or risk for having a disease associated with oxidative modification of a protein, said second protein fraction lacking nitrotyrosine, wherein determination of the presence of nitrotyrosine in said first protein fraction and the absence of nitrotyrosine in said second protein fraction indicates risk for having or presence of a disease in the first subject; and
determining the protein from which said at least one peptide that includes a nitrotyrosine residue is derived, and therefrom identifying a protein that is oxidatively modified in the disease.

17. A method of identifying a suitable agent for treating a disease associated with oxidative modification of a protein, comprising:
comparing (i) a first mass spectrum of all or a portion of a first protein fraction of a biological sample derived from a subject having or being at risk for having a disease associated with oxidative modification of a protein, prior to contacting said sample with a candidate agent, said first protein fraction comprising at least one peptide that includes a nitrotyrosine residue, to (ii) a second mass spectrum of all or a portion of a second protein fraction of a biological sample derived from the subject subsequent to contacting said sample with the candidate agent, wherein determination of a decreased level of nitrotyrosine in said second mass spectrum relative to said first mass spectrum indicates the agent reduces oxidative protein modification.

18. A method of identifying a suitable agent for treating a disease associated with oxidative modification of a protein, comprising:
comparing at least one biological activity of a protein identified according to the method of claim 16 in the absence of a candidate agent to the biological activity of the protein in the presence of the candidate agent, wherein an alteration of said activity indicates suitability of the agent for treating a disease associated with oxidative protein modification.

19. A method for identifying oxidative modification of a proteome, comprising:
generating a mass spectrum of all or a portion of a protein fraction derived from a biological sample, the protein fraction comprising a plurality of proteins that each contain a nitrotyrosine residue, wherein determination of nitrotyrosine in said sample indicates the proteins are oxidatively modified.
